(19) United States
(12) Patent Application Publication
(10) Pub. No.: US 2026/0275276 A1
(43) Pub. Date: Sep. 17, 2026

(54) IMMORTALIZATION OF AMNION-DERIVED MULTIPOTENT PROGENITOR CELL AND USES THEREOF

(71) Applicant: NOVEOME BIOTHERAPEUTICS, INC., Pittsburgh, PA (US)

(72) Inventors: Hedvika DAVIS, Pittsburgh, PA (US); Randall RUPP, Swanton, VT (US); Ashley ELLIS, Pittsburgh, PA (US); Christine SMITH, Cranberry Twp., PA (US)

(21) Appl. No.: 19/168,512

(22) PCT Filed: Mar. 22, 2024
(86) PCT No.: PCT/US2024/021177

§ 371 Date: Sep. 24, 2025

Related U.S. Application Data
(60) Provisional application No. 63/454,522, filed on Mar. 24, 2023.
(60) Provisional application No. 63/530,762, filed on Aug. 4, 2023.

(51) Int. Cl. C12N 5/073

(52) U.S. Cl. CPC C12N 5/0605; C12N 2510/04

(57) ABSTRACT

An immortalized amnion-derived multipotent progenitor (AMP) cell or cell line and methods of producing same. The immortalized AMP cells secrete a secretome (ST266*), in a highly scalable, cost-effective manner. The immortalized AMP cells may be proliferated in serum-free medium without the presence of HSA and EGF. Provided herein are compositions obtained from the secretome collected from at least one immortalized AMP cell.

### CROSS-REFERENCE TO RELATED APPLICATIONS

This application is a National Stage of PCT/US2024/021177 entitled IMMORTALIZATION OF AMNION-DERIVED MULTIPOTENT PROGENITOR CELL AND USES THEREOF, filed on Mar. 22, 2024, in which claims priority to and the benefit of U.S. Provisional Application No. 63/454,522, filed on Mar. 24, 2023, entitled IMMORTALIZATION OF AMNION-DERIVED MULTIPOTENT PROGENITOR CELL AND USES THEREOF, and U.S. Provisional Application No. 63/530,762, filed on Aug. 4, 2023, entitled IMMORTALIZATION OF AMNION-DERIVED MULTIPOTENT PROGENITOR CELL AND USES THEREOF, each of which is incorporated by reference herein in its entirety.

### TECHNICAL FIELD

The present disclosure relates generally to the development of immortalized cell lines and methods of producing the same.

### BACKGROUND

Amnion-derived multipotent progenitor cells (AMP cells) are isolated from the amnion derived epithelium of full-term placentas. The non-immunogenic epithelial cells are selected based on their ability to attach and proliferate in serum-free conditions without the presence of animal-derived components. The selected subpopulation of AMP cells produces various factors found in amniotic fluid with a multitude of regenerative and protective capabilities. Obtaining full-term placentas, which are normally discarded after birth, is difficult due to lack of a sufficient number of donors and regulations. AMP cells, as most primary epithelial cells, undergo only a limited number of divisions in culture before they become senescent due to an increase in inhibitors of cyclin-dependent kinases and telomere shortening, leading to loss of telomeric region.

Immortalized cells that can reproduce substantially indefinitely may provide a more cost-effective, easy to use alternative to primary cells.

### SUMMARY

The present disclosure addresses many of the shortcomings of obtaining and utilizing non-immortalized amnion-derived multipotent progenitor (AMP) cells. The immortalized AMP cell and the methods of immortalizing an AMP cell of the present disclosure provide an AMP cell line that may be cultured substantially indefinitely or indefinitely, allowing the cells and any secretions provided therefrom to be produced in a highly scalable, cost-effective manner.

The present disclosure provides immortalized AMP cells capable of proliferation in serum-free conditions without the presence of animal-derived components. According to certain aspects, AMP cells are transfected with hTERT and HPV-16 E6/E7 individually or in combination using a transposon-transposase system to produce immortalized AMP cell lines.

Non-immortalized AMP cells were found to secrete hundreds of bioactive molecules, many of which are anti-inflammatory, and all of which are at physiological concentrations (pg/mL to ng/ml), i.e., the AMP secretome or ST266. Compared to ST266, the secretome of the immortalized AMP cells of the present disclosure, ST266*, demonstrates identical, nearly identical, or improved concentrations of the bioactive molecules.

### STATEMENT REGARDING DEPOSIT

The immortalized monoclonal AMP cell lines of the present disclosure will be deposited in the American Type Culture Collection (“ATCC”), if required by the United States Patent and Trademark Office per 37 CFR § 1.809(a), during the pendency of this application. As necessary, the cell lines will be available to the public as of the issue date of a patent on this subject matter, will be replaced if the culture mutates or becomes nonviable, and will be maintained for a term of 30 years, or five years after the last request for such deposit, or for the effective life of the patent, whichever is longest.

### BRIEF DESCRIPTION OF THE DRAWINGS

It is to be understood that both the foregoing summary and the following drawings and detailed description may be exemplary and may not be restrictive of the aspects of the present disclosure as claimed. Certain details may be set forth in order to provide a better understanding of various features, aspects, and advantages of the invention. However, one skilled in the art will understand that these features, aspects, and advantages may be practiced without these details. In other instances, well-known structures, methods, and/or processes associated with methods of practicing the various features, aspects, and advantages may not be shown or described in detail to avoid unnecessarily obscuring descriptions of other details of the invention.

FIG. 1 provides a graph of the number of unique proteins identified within various molecular weight ranges derived from a multiple advanced HPLC Mass Spectrometry proteomic analysis of an ST266 composition.

FIG. 2A is a phase contrast image of un-transfected AMP cells at day 18 in culture at 10× magnification (scale bar indicates 100 μm).

FIG. 2B is a phase contrast image of AMP cells transfected using microspheres at day 18 in culture and 10 days after the transfection at 10× magnification.

FIG. 2C is a phase contrast image of AMP cells transfected using microspheres at day 38 in culture and 20 days after the transfection at 10× magnification.

FIG. 3A is a phase contrast image of primary un-transfected AMP cells at day 10 in culture at 10× magnification (scale bar indicates 100 μm).

FIG. 3B is a phase contrast image of immortalized monoclonal AMP cells of the present disclosure at day 200 in culture at 10× magnification.

FIG. 3C is a phase contrast image of immortalized monoclonal AMP cells of the present disclosure at day 330 in culture at 10× magnification.

FIGS. 4A and 4B are cell growth curves of primary AMP cells and immortalized polyclonal AMP culture (FIG. 4A) and immortalized monoclonal cell lines originating from the transfected polyclonal AMP cells (FIG. 4B) of the present disclosure.

FIG. 5A shows a phase contrast image of non-immortalized AMP cells cultured with HSA and EGF at 10× magnification. FIG. 5B is a phase contrast image of non-immortalized AMP cells without HSA and without EGF at 10× magnification. FIG. 5C demonstrates a phase contrast image of PB-E6-E7-PB-TERT—clone 1 at 235 days in culture, grown and passaged 30 days without EGF (scale bar indicates 100 μm). FIG. 5D demonstrates a phase contrast image of PB-E6-E7-PB-TERT—clone 8 at 235 days in culture, grown and passaged 30 days without EGF. FIG. 5E demonstrates a phase contrast image of PB-E6-E7-PB-TERT—clone 1 in alternative medium at 235 days in culture, grown and passaged in the presence of HSA and EGF for 30 days. FIG. 5F demonstrates a phase contrast image of PB-E6-E7-PB-TERT—clone 1 in alternative medium at 235 days in culture, grown and passaged without HSA and EGF for 30 days.

FIG. 6A is an image of a 2% agarose gel comprising PCR products of transgenes inducing immortalization inserted into genomic DNA from immortalized AMP cells of the present disclosure separated by gel electrophoresis, stained with ethidium bromide, and imaged using UVP BioImaging System.

FIGS. 6B & 6C provide images of immortalized AMP cells of the present disclosure expressing hTERT, HPV-16-E6 and E7 proteins at 235 days in culture, using the Zeiss LSM 880 confocal laser scanning microscope at 40× magnification (scale bar represents 50 μm).

FIG. 7 provides a table of the secretome compositions secreted by immortalized AMP cell clones (ST266*) at day 125 and primary AMP cells (ST266).

FIGS. 8A through 8J are serum analyses from ST266* treated mice using a MSD Cytokine Assay for biomarkers interferon γ (IFN-γ) (FIG. 8A), interleukin-1β (IL-1β) (FIG. 8B), interleukin-2 (IL-2) (FIG. 8C), interleukin-4 (IL-4) (FIG. 8D), interleukin-5 (IL-5) (FIG. 8E), interleukin-6 (IL-6) (FIG. 8F), interleukin-10 (IL-10) (FIG. 8G), interleukin-12p70 (IL-12p70) (FIG. 8H), chemokine keratinocyte chemoattractant (KC)/human growth-regulated oncogene (KC/GRO) (FIG. 8I) and tumor necrosis factor α (TNF-α) (FIG. 8J)

FIG. 9 is a cell growth curve of primary AMP cells and immortalized AMP cells of the present disclosure.

### DETAILED DESCRIPTION

### Definitions

In this disclosure, certain terms are used which shall have the meanings set forth as follows.

Various aspects of the methods may be described and illustrated with reference to one or more exemplary implementations. As used herein, the term “exemplary” means “serving as an example, instance, or illustration,” and should not necessarily be construed as preferred or advantageous over other variations of the devices, systems, or methods disclosed herein. “Optional” or “optionally” means that the subsequently described event or circumstance may or may not occur, and that the description includes instances where the implementation occurs and instances where it does not.

It must also be noted that as used herein and in the aspects, the singular forms “a”, “an”, and “the” such as “a” cell culture, “an” immortalized cell, and “the” secretome include the plural reference unless the context clearly dictates otherwise.

As used herein, the term “and/or” includes any and all combinations of one or more of the associated listed items. Likewise, as used herein, a term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Words such as “then,” “next,” etc. are not intended to limit the order of the steps; these words are simply used to guide the reader through the description of the methods.

The terms “comprises”, “comprising”, “including”, “having”, and “characterized by”, may be inclusive and therefore specify the presence of stated features, elements, compositions, steps, integers, operations, and/or components, but do not preclude the presence or addition of one or more other features, integers, steps, operations, elements, components, and/or groups thereof. Although these open-ended terms may be to be understood as a non-restrictive term used to describe and claim various aspects set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, described herein also specifically includes embodiments consisting of, or consisting essentially of, such recited compositions, materials, components, elements, features, integers, operations, and/or process steps. In the case of “consisting of”, the alternative embodiment excludes any additional compositions, materials, components, elements, features, integers, operations, and/or process steps, while in the case of “consisting essentially of”, any additional compositions, materials, components, elements, features, integers, operations, and/or process steps that materially affect the basic and novel characteristics may be excluded from such an embodiment, but any compositions, materials, components, elements, features, integers, operations, and/or process steps that do not materially affect the basic and novel characteristics may be included in the embodiment.

Any method steps, processes, and operations described herein may not be construed as necessarily requiring their performance in the particular order discussed or illustrated, unless specifically identified as an order of performance. It is also understood that additional or alternative steps may be employed, unless otherwise indicated.

In addition, features described with respect to certain example embodiments may be combined in or with various other example embodiments in any permutational or combinatory manner. Different aspects or elements of example embodiments, as disclosed herein, may be combined in a similar manner. The term “combination”, “combinatory,” or “combinations thereof”′ as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included may be combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

While specific aspects of the disclosure have been provided hereinabove, the disclosure may, however, be embodied in many different forms and should not be construed as necessarily being limited to only the embodiments disclosed herein. Rather, these embodiments may be provided so that this disclosure is thorough and complete, and fully conveys various concepts of this disclosure to skilled artisans.

Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

All numerical quantities stated herein may be approximate, unless stated otherwise. Accordingly, the term “about” may be inferred when not expressly stated. The numerical quantities disclosed herein may be to be understood as not being strictly limited to the exact numerical values recited. Instead, unless stated otherwise, each numerical value stated herein is intended to mean both the recited value and a functionally equivalent range surrounding that value. At the very least, and not as an attempt to limit the application of the doctrine of equivalents to the scope of the claims, each numerical value should at least be construed in light of the number of reported significant digits and by applying ordinary rounding processes. Typical exemplary degrees of error may be within 20%, 10%, or 5% of a given value or range of values. Alternatively, the term “about” refers to values within an order of magnitude, potentially within 5-fold or 2-fold of a given value. Notwithstanding the approximations of numerical quantities stated herein, the numerical quantities described in specific examples of actual measured values may be reported as precisely as possible. Any numerical values, however, inherently contain certain errors necessarily resulting from the standard deviation found in their respective testing measurements.

All numerical ranges stated herein include all sub-ranges subsumed therein. For example, a range of “1 to 10” or “1-10” is intended to include all sub-ranges between and including the recited minimum value of 1 and the recited maximum value of 10 because the disclosed numerical ranges may be continuous and include every value between the minimum and maximum values. Any maximum numerical limitation recited herein is intended to include all lower numerical limitations. Any minimum numerical limitation recited herein is intended to include all higher numerical limitations.

Features or functionality described with respect to certain example embodiments may be combined and sub-combined in and/or with various other example embodiments. Also, different aspects and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, may be at least partially performed via at least one entity or actor in any manner.

While particular embodiments have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses, cell lines, mediums, and methods described herein, including alternatives, variants, additions, deletions, modifications, and substitutions. This application including the appended claims is therefore intended to cover all such changes and modifications that may be within the scope of this application.

The term “cell line” as used herein, refers to individual cells, harvested cells, and cultures containing the cells, so long as they are derived from cells of the cell line referred to. A cell line is said to be “continuous,” “immortal,” or “stable” if the line remains viable over a prolonged time, typically at least six months. To be considered a cell line, as used herein, the cells must remain viable for at least 40 passages in the absence of exogenous growth factors. A “cell strain,” in contrast, refers to cells that do not remain viable over a prolonged time in the absence of exogenous growth factors.

As used herein, the term “isolated” refers to material removed from its original environment and is thus altered “by the hand of man” from its natural state.

As used herein, the term “substantially purified” means a population of cells substantially homogeneous for a particular marker or combination of markers. By substantially homogenous is meant at least 90%, and preferably 95% homogenous for a particular marker or combination of markers.

The term “protein marker” as used herein means any protein molecule characteristic of a cell or cell population. The protein marker may be located on the plasma membrane of a cell or in some cases may be a secreted protein.

The term “enriched” as used herein means to selectively concentrate or to increase the amount of one or more materials by elimination of the unwanted materials or selection and separation of desirable materials from a mixture (i.e., separate cells with specific cell markers from a heterogeneous cell population in which not all cells in the population express the marker).

The term “placenta” as used herein refers to both pre-term and term placenta.

As used herein, the term “extraembryonic tissue” means tissued located outside the embryonic body, which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois, and amniotic fluid (including all components contained therein). Extraembryonic tissue and cells derived therefrom have the same genotype as the developing embryo.

The term “immortalized” as used herein means a population of cells, for example AMP cells, that have been manipulated such that they are capable of a significantly greater number of population doublings than the population of cells was capable of prior to the manipulation.

The term “immortalized cell line” as used herein means a cell line that can replicated and be maintained substantially indefinitely in in vitro cultures under conditions that promote growth, preferable at least over a period of a year or years. The term “substantially indefinitely”, as used herein, is meant the “immortalized cell line” is capable of at least 70 passages.

The term “Immortalized Amnion-derived Multipotent Progenitor cells” or “i-AMP cells” as used herein means a population of AMP cells that has been manipulated such that it is capable of greater than 70 population doublings.

The term “passage” refers to a cell culture technique in which cells growing in culture that have attained confluence or are close to confluence in a tissue culture vessel are removed from the vessel, diluted with fresh culture media (i.e., diluted 1:5) and placed into a new tissue culture vessel to allow for their continued growth and viability. For example, cells isolated from the amnion are referred to as primary cells. Such cells may be expanded in culture by being grown in the growth medium described herein. When such primary cells are subcultured, each round of subculturing is referred to as a passage. As used herein, “primary culture” means the freshly isolated cell population, the number of times that a cell population has been removed from the culture vessel and undergone a subculture (passage) process, in order to keep the cells at a sufficiently low density to stimulate further growth.

As used herein, the terms “culture media”, “culture medium”, “media”, and “medium” may be used interchangeably.

The term “in vitro” as used herein denotes outside, or external to, animal or human body. The term “in vitro” as used herein should be understood to include “ex vivo”. The term “ex vivo” typically refers to tissues or cells removed from an animal or human body and maintained or propagated outside the body, e.g., in a culture vessel from propagation, e.g., ex vivo propagation, of a cell that has been removed from its native environment.

As used herein, a “gene” is the segment of DNA involved in producing a polypeptide chain; it includes regions preceding and following the coding region, as well as intervening sequences (introns) between individual coding segments (exons). In recombinant DNA technology, genes inserted into expression vectors typically do not include introns.

The term “transposon” refers to DNA sequences that are capable of moving from one location to another in a genome. The term “transposon” as used herein should be understood to include “transposable elements” found in both prokaryotic and eukaryotic organisms.

A DNA transposon system, such as the PiggyBac transposon system described herein, may be composed of a transposon and transposase. The transposon may include a gene of interest and inverted terminal repeats (ITRs) having binding sites for transposase. Transposase is a transposition enzyme, which binds to ITRs at both ends of a transposon and catalyzes its movement to specific sites of the genome by a cut-and-paste transposition mechanism. PiggyBac transposases integrate the gene of interest into specific TTAA chromosomal sites. Among eukaryotic DNA transposon systems, only PiggyBac is known to specifically integrate at TTAA sites. Cells may then be transiently re-transfected with the PiggyBac transposase which removes the genes of interest from the genome, leaving no sequences or mutations behind. The 5′ TTAA overhands created at the excision site may anneal, thus repairing the double-stranded break without leaving any footprint. This seamless reversible modification of the genome eliminates predisposition toward tumorigenesis or unexpected cellular behavior, providing benefits for the production of immortalized cell lines of the present disclosure and the use of ST266* for clinical applications.

The term “transfected” as used herein refers to the stable transfection or the transient transfection of the cell of the present disclosure.

The term “stable transfection” or “stably transfected” refers to the introduction and integration of foreign nucleic acid sequence into the genome of the transfected cell. The term “stable transfectant” refers to a cell that has stable integrated foreign DNA into the genomic DNA.

The term “target DNA sequence” is a predetermined region within the genome of a cell which is targeted for precise and stable insertion of a gene of interest. As a non-limiting example, the cut and paste transposition mechanisms of the present disclosure may enable the precise and stable insertion of a gene of interest. Target DNA sequences include structural genes (i.e., DNA sequences encoding polypeptides including in the case of eucaryotes, introns and exons), regulatory sequences such as enhancers sequences, promoters, and the like, and other regions within the genome of interest. A “target DNA sequence” may also be a sequence which, when targeted by a vector, has no effect on the function of the host genome.

The term “inserted into a target DNA sequence” as used herein means insertion of the gene of interest into genomic DNA by a cut and paste transposition mechanism. For example, during transposition, the PiggyBac transposases may recognize specific ITRs located on each end of the transposon. After ITR recognition, the PiggyBac transposase may excise, or cut, the transposon from the vector and integrates, or pastes, the transposon into TTAA sites in the genomic DNA of the mammalian cells.

As used herein, the term “Amnion-derived Multipotent Progenitor cell” or “AMP cell” means a specific population of cells that are amnion epithelial cells derived from the amnion of a placenta. AMP cells have the following characteristics. They secrete at least one or more of the cytokines VEGF, Angiogenin, PDGF and TGFβ2 and at least one or more of the MMP inhibitors TIMP-1 and TIMP-2. The immortalized and non-immortalized AMP cells of the present disclosure may not have been cultured in the presence of any non-human animal-derived products or substances. As such, certain of the immortalized cells and the cell products derived from them, as disclosed herein, are suitable for human clinical use as they are not xeno-contaminated. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are selected, have no reaction with an antibody to the stem/progenitor cell marker c-kit (CD117), and minimal to no reaction with an antibody to the stem/progenitor cell marker Thy-1 (CD90).

As used herein, “conditioned medium” may be understood to be a medium in which a specific cell or population of cells has been cultured, and then removed. When cells are cultured in a medium, they may secrete cellular factors that can provide support to or affect the behavior of other cells. Such factors include, but are not limited to, hormones, cytokines, extracellular matrix (ECM), proteins, vesicles, antibodies, chemokines, receptors, inhibitors, and granules. The medium containing the cellular factors is the conditioned medium, also referred to herein as the “secretome”.

As used herein, the term “alternative medium” means a medium including one or more amino acids or salts thereof, one or more vitamins, one or more nucleotides, and one or more trace elements but not including proteins and peptides.

As used herein, “feeder” means a cell unable to divide, which provides extracellular secretions to help another cell to proliferate. A “feeder” cell releases growth factors to culture medium. A “feeder” cell helps progenitor cells grow and differentiate. Thus, the term “feeder-free” refers to a culture medium without “feeder” cells.

The non-immortalized AMP cells and immortalized AMP cells of the present disclosure secrete a unique combination of physiologically relevant molecules in a physiologically relevant temporal manner into the extracellular space or into surrounding culture medium. The term “ST266” refers to conditioned medium that has been derived from non-immortalized AMP cells, while the term “ST266*” refers to conditioned medium that has been derived from immortalized AMP cells. Each of ST266 and ST266* can refer to the crude conditioned medium or can refer to a composition obtained from the crude conditioned medium after it has been subjected to further processing as described herein.

The term “animal-free,” when used in reference to certain compositions, growth conditions, culture media, etc., described herein, means that no non-human animal-derived materials, such as bovine serum, proteins, lipids, carbohydrates, nucleic acids, vitamins, etc., are used in the preparation, growth, culturing, expansion, storage or formulation of AMP cells or the immortalized AMP cells of the present disclosure, and their secreted products or compositions thereof. By “no non-human animal-derived materials” is meant that the materials have never been in or in contact with a non-human animal body or substance, so they are not xeno-contaminated. Only clinical grade materials, such as recombinantly produced human proteins, are used in the preparation, growth, culturing, expansion, storage and/or formulation of non-immortalized AMP cells and immortalized AMP cells, as well as ST266 and ST266*.

The term “serum-free” when referring to certain compositions, growth conditions, culture media, etc. described herein, is meant that no non-human animal derived serum is used in the preparation, growth, culturing, expansion, storage, or formulation of the compositions or processes of the present disclosure.

In the following description, certain details are set forth in order to provide a better understanding of various aspects of the methods disclosed herein. However, one skilled in the art will understand that these aspects may be practiced without these details and/or in the absence of any details not described herein. In other instances, well-known structures, methods, and/or techniques associated with methods of practicing the various embodiments may not be shown or described in detail to avoid unnecessarily obscuring descriptions of other details of the various aspects.

While particular aspects have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications, and substitutions. This application including the appended aspects is therefore intended to cover all such changes and modifications that are within the scope of this application.

Unless defined otherwise, all technical and scientific terms used herein have the same meanings as commonly understood by one of ordinary skill in the art.

### Aspects of the Present Disclosure

The present disclosure provides a method for the immortalization of amnion-derived multipotent progenitor (AMP) cells and generation of immortalized monoclonal cell lines. Human amnion epithelial cells (hAECs) are derived from the innermost layer of the placenta, i.e., the layer closest to the fetus. Once the cells are isolated from the amnion, they are used to select and culture AMP cells. In culture conditions, amnion-derived multipotent progenitor cells exhibit stem cell-like plasticity and differentiation potential (M. Toshio et al., 2005). Various methods for isolating cells from the amnion of a human placenta are described in the art (see, for example, US2003/0235563, US2005/0124003, US2006/0222634, US2006/0078993, US2004/0048372, US2003/0032179, WO2006/105152, WO2000/73421, U.S. Provisional Application Nos. 60/666,949, 60/699,257, 60/742,067, 60/813,759, and U.S. Pat. No. 7,045,148). Thus, those of skill in the art may understand methods for isolating epithelial cells from the amnion. Briefly, AMP cell compositions are prepared using the steps of a) recovery of the amnion from the placenta, b) dissociation of the epithelial cells from the amniotic membrane, c) culturing of the dissociated epithelial cells, d) selecting the adherent cells (the AMP cells) and discarding the non-adherent cells from the cell culture, and optionally e) further proliferation of the cells, optionally using additional additives and/or growth factors. Isolation methods may include peeling of the chorion from the amnion and separation of hAECs from the underlying basement membranes via enzymatic digestion by an enzymatic solution such as trypsin/EDTA, dispase II, and detergents.

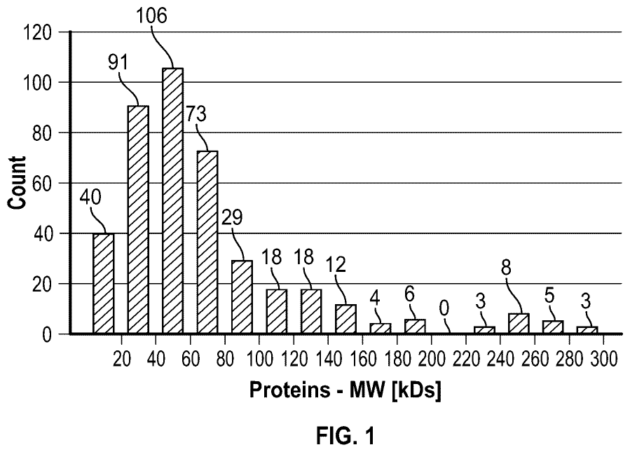

The non-immunogenic epithelial cells are selected based on their ability to attach and proliferate in serum-free conditions without the presence of animal-derived components. The non-immortalized AMP cells secrete a secretome, ST266, that contains approximately 1700 bioactive molecules at physiological concentrations (pg/mL to ng/ml) (FIG. 1).

Since primary or non-immortalized AMP cells have limited proliferation capabilities, the cells typically lose proliferation potential after about 60-80 days in culture or about four (4) to five (5) passages in culture. Moreover, human placentas are difficult to obtain and do not provide a consistent source of material. As a non-limiting example, human placentas are not readily available to obtain a sufficient number of AMP cells for mass production of ST266 for therapeutic use. As another non-limiting example, obtaining human placentas results in genetic variability, which may negatively affect the secretions of the AMP cells obtained from the placentas. The present disclosure provides immortalized AMP cells allowing for a sufficient system to produce a novel secretome therefrom, ST266*, on a large scale. The immortalization of AMP cells further reduces genetic variability found among placental donors.

Many epithelial cells, unlike most other cell types, may senesce in at least two steps. The first step occurs when various signals, such as physiological stress, activate the p16INK4a/Rb pathway, leading to inhibition of the cell cycle. This initial growth arrest occurs before the telomeres become critically short. The retinoblastoma protein Rb and p16^{INK4a} have known roles as inhibitors of the cell cycle progression from G1 to S phase. Accordingly, overcoming of Rb/p16INK4a mediated inhibition of the cell cycle is the initial step required for immortalization of epithelial cells. However, with each division, and in the absence of telomerase activity, telomeres of the cells are shortened until they reach a critical point leading to cell cycle arrest and senescence. The loss of telomeric regions may be avoided through the second step, by the introduction of exogenous telomerase (H. J. Muller, 1938; B. McClintock, 1942; A G. Bodnar et al., 1998; T M. Nakamura et al., 1998; J W. Shay et al., 1994; T. Kiyono et al., 1998; R D Ramirez et al., 2004).

To immortalize isolated AMP epithelial cells, their genome may be stably modified. To achieve the first immortalization step, i.e., overcoming inhibition of the cell cycle, the genome may be modified by stable integration of E6 and E7 transgenes from the human papillomavirus (HPV 16). Both proteins induce cell cycle progression by inhibition of negative regulations of the cell cycle: P53 and Rb proteins. P53 is inactivated by E6 and the Rb/p16^{INK4a} pathway by E7 (Dyson N. et al, 1989, Cheng, S. et al., 1995, Nevins, J., 1992, Shay et al., 1994, Eun-Kyoung Yim, 2005). A second immortalization step may involve the introduction of telomerase, which is essential for replication once cells begin to exhaust their telomeres below a critical minimal length.

Telomerase may be introduced to the AMP cells of the present disclosure through the ectopic expression of human telomerase reverse transcriptase (hTERT), allowing cells to repair telomeres and avoid senescence. While differentiated somatic cells have a limited life span before reaching senescence, the introduction of specific viral oncogenes can lead to cell immortalization. Ectopic expression of hTERT may provide one or more of the following benefits: prolonged cellular life, improved growth characteristics, a stabilized karyotype, a maintained original cellular phenotype, and improved differentiation potential. (Zhou et al, 2013). Exogenous expression of HPV-16 E6/E7 may be required to inactivate the retinoblastoma (Rb)/p16^{INK4a} tumor-suppression pathway to allow cell cycle progression.

According to the present disclosure, the immortalization of AMP cells may be achieved by a combination of hTERT and HPV-16 E6/E7, wherein ectopic expression of hTERT alone and HPV-16 E6/E7 alone may result in extension in cellular lifespan.

The hTERT and HPV-16 E6/E7 sequences may be delivered to AMP cells via a number of different systems, including, but not limited to, a transposon-transposase system, viral vectors, or traditional plasmid vectors.

Transposons are promising elements for gene integration and constitute a significant component of repetitive sequences in eukaryotes. DNA transposons move between genomic sites via a cut and paste mechanism. (Kim et al, 2011). Moreover, a transposon system is highly efficient and site specific compared to other methods of gene delivery and is therefore more suitable for use in human therapeutical applications compared to viral or traditional plasmid vectors.

While plasmid delivery is possible, a transposon system is preferable, as plasmid delivery of the gene of interest typically results in a smaller percentage of stable integration and most of the delivered DNA is degraded, especially in fast dividing cells. Unlike transfection using plasmids, transposons achieve high and stable expression of the therapeutic gene.

During transposition, only the genes of interest are inserted into the host genome without any bacterial sequences from the plasmid vector backbone. A transposon system also eliminates potential risks associated with viral vectors, such as infection risks and immune concerns. Viruses may have high immunogenicity and inclination to target active genes and could induce tumorigenesis, and once genes are inserted, they have a tendency to be silenced. Furthermore, transposon systems are simpler and more convenient, as they do not require packing and allow for stable integration in the cellular genome.

Accordingly, the hTERT and/or HPV-16 E6/E7 sequences may be delivered to AMP cells via a transposon system. As such, the immortalized cells of the present disclosure are not tumorigenic.

Epithelial cells are difficult to immortalize and require at least two steps to achieve immortalization. First, activation through the G1 phase of the cell cycle is necessary to overcome certain cellular checkpoints. Furthermore, isolating AMP cells from a placenta adds additional complexities, as placental epithelial cells have evolved to have protective mechanisms, which are difficult to overcome, to protect the developing fetus. Conventional methods of potential AMP cell immortalization using a plasmid or a retrovirus are unable to provide sufficient cell numbers for experimentation, were unable to fully immortalize the cells, used heterogeneous population cultured in serum containing medium and did not result in a uniform monoclonal cell line with primary cell characteristics, unlimited growth, and reproducible results, unlike the immortalized AMP cells of the present disclosure. Attempts using a CRISPR/Cas9 method have failed as the telomerase promoter region forms structures that endonucleases cannot enter. Attempts using viral transfection result in infection risks and immune response concerns. Furthermore, the use of bacterial plasmids involves use of bacterial DNA and results in xeno-contamination. Thus, the methods of the present disclosure, that include use of a transposon system to transfect AMP cells resulting in immortalized cells and immortalized monoclonal cell lines without xeno-contamination and with stable integration into the genome, have not been successful in the prior art.

The transposons may remain stably present in the cellular genome after integration into the genome. Accordingly, immortalized AMP cells of the present disclosure may comprise transposons or may not comprise transposons.

Transfection may be accomplished using commercial kits, such as those available from Bio-Rad (2000 Alfred Nobel drive, Hercules, Calif. 94547, USA) and Invitrogen (Life Technology, 3175 Staley Road, Grand Island, N.Y. 14072, USA). Commonly used reagent-based transfection methods comprise the use of lipids, calcium phosphate, cationic polymers, DEAE-dextran, activated dendrimers and magnetic beads. Instrument-based methods comprise electroporation and micro-injection.

Transfection may also be accomplished by liposome transfection, which is a technique for inserting genetic material into cells using liposomes. That is, cationic lipids are used to form liposomes, which take up nucleic acids. The positive charge of the liposomes and negative charge of the nucleic acids allow the two to form a complex, which can then enter the cell through endocytosis. An exemplary reagent useful for liposome transfection is Lipofectamine™ 2000 Transfection Reagent (Thermo Fisher Scientific, Waltham, MA, USA). The transfection may be accomplished using microspheres, such as described in U.S. Pat. No. 9,339,465 (Brown et al.), which is hereby incorporated by reference in its entirety. The microsphere method is a simple and effective method of delivering nucleic acids into cells without damaging effects associated with other commonly used transfection reagents. Microspheres may be composed of nucleic acids dissolved in aqueous-albumin solution, combined with water-soluble polymers polyvinyl pyrrolidone (PVP) and polyethylene glycol (PEG). These water-soluble polymers do not form a substantial part of the microspheres but aid in their preparation.

The immortalized AMP cells of the present disclosure may be transfected by Lipofectamine™ 2000 or microspheres. In one aspects, the microspheres may be prepared by combining a pair of site-specific recombination sequences with a polymer solution, and incubating the solution for 30 minutes at 70° C. followed by cooling to 23° C. In one aspect, the polymer solution is a 25% polymer solution in a 1:2 v/v volumetric ratio. The polymer solution may comprise in another aspects, the 25% polymer solution may comprise 12.5% PVP (40,000 Daltons) and 12. % PEG in 1M sodium acetate at pH 5.5.

The immortalized AMP cells of the present disclosure transfected by microspheres may appear healthier and may have higher proliferation rates immediately after transfected compared to cells transfected by another method of transfection such as Lipofectamine™.

A DNA molecule comprising transposons and hTERT and/or HPV-16 E6/E7 genes under the control of a suitable promoter may be a plasmid form when it is used for the transfection step. Exemplary transposon systems include PiggyBac, Sleeping Beauty, Tol2, Mos1, and Frog Prince.

According to certain aspects, the PiggyBac transposon system may be used. PiggyBac is advantageous over other transposon systems as it is precisely excised when a transposition takes place, and it does not leave a DNA footprint. Among eukaryotic DNA transposons, only piggyBac is known to specifically integrate at TTAA sites. It has been shown that 97% of the insertion sites occurred within introns in human and mice. Cells may then be transiently re-transfected with the piggyBac transposase which will remove the genes of interest from the genome, leaving no sequences or mutations behind. This reversible modification of the genome may eliminate a predisposition toward tumorigenesis or unexpected cellular behavior. The TTAA integration sites used by piggyBac are repaired to its original sequence on excision. As such, transposons may be removed from the host genome without changing any nucleotide sequence. (B. McClintock, 1950; A. Lacoste et al., 2009; Wilson et al., 2007; Q. Chen et al., 2020; L. Pray, 2008; ER. Burnight, et al., 2012; KA. Skipper et al., 2013).

The use of transposons is well-known in the art but not for the immortalization of AMP cells. A paper by Ivics, Z. and Izsvak, Z. extensively reviews transposons and their use (Mobile DNA 1:25-39 (2010)). Further, Kim, A. and Pyykko, 2011 describes the use and mechanisms of PiggyBac and other transposons. Transposition efficiency is dependent on the availability of transposon and transposase in cells. (Chiung-Yuan Wu, et al., 2006). According to certain aspects, the cells of the present disclosure may comprise a transposon. The transposase containing plasmid may be degraded within 72 hours. Thus, the immortalized AMP cells of the present disclosure may not include transposase.

Fluorescent proteins such as mCherry may be present in the plasmid encoded transposase as a reporter tag, which may aid in the identification of the transposon's stable incorporation of genes of interest into the cellular genome. The plasmid may only result in transient gene delivery of the transposase and/or the mCherry without stable incorporation to the genome. The transposase containing plasmid may be degraded within 72 hours. Thus, the immortalized AMP cells of the present disclosure may not comprise fluorescent proteins.

According to the methods of the present disclosure, the immortalized AMP cells may also be generated without fluorescent proteins or selection markers, wherein the plasmid encoded transposase consists essentially of the genes of interest without any modifications such as a selection marker or reporter marker (e.g., fluorescent protein). Thus, the secretome (ST266*) of the present disclosure may be free, substantially free, essentially free, or completely free of selection markers or fluorescent proteins.

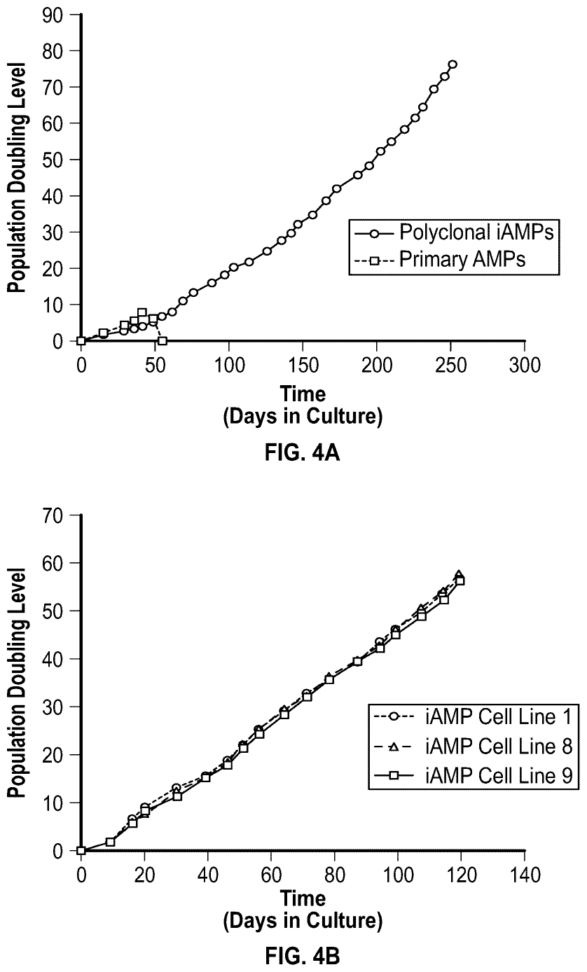

Immortalized AMP cells of the present disclosure may be cultured without antibiotics. This avoids the toxic effect antibiotics may have on cells and the undesirable presence of antibiotic in the therapeutic secretome (ST266*). Instead of antibiotic selection, growth advantage selection may be utilized, wherein the immortalized cells exhibit increasingly faster proliferation and expand throughout the surface area of a plate (FIGS. 4A and 4B), while untransformed cells become enlarged and undergo a growth arrest.

The immortalized AMP cells of the present disclosure are able to undergo greater than 20 population doublings, preferably greater than 50 population doublings, and most preferably greater than 70 population doublings.

The present disclosure provides a composition comprising a population of immortalized amnion-derived multipotent progenitor cells, wherein the immortalized AMP cells are capable of greater than 12 population doublings, such as greater than 20 population doublings, and most preferably greater than 70 population doublings.

In the following description of immortalized AMP cell lines, PB refers to Piggbac, E6 and E7 refer to stable expression of E6 and E7 proteins from HPV-16, and TERT refers to stable expression of hTERT. The present disclosure provides immortalized AMP cell lines, wherein the immortalized monoclonal AMP cell lines are preferably clones of single cells expressing PB-E6-E7-PB-TERT, including, but not limited to, clones: PB-E6-E7-PB-TERT—clone 1, PB-E6-E7-PB-TERT—clone 2, PB-E6-E7-PB-TERT—clone 3, PB-E6-E7-PB-TERT—clone 4, PB-E6-E7-PB-TERT—clone 5, PB-E6-E7-PB-TERT—clone 6, PB-E6-E7-PB-TERT—clone 7, PB-E6-E7-PB-TERT—clone 8, PB-E6-E7-PB-TERT—clone 9, and PB-E6-E7-PB-TERT—clone 10.

The immortalized AMP cells of the present disclosure may provide a secretome (ST266*) having identical, nearly identical, substantially the same, or improved therapeutic properties and efficacy as the secretome ST266 disclosed and claimed in U.S. Patent Publication 2020/0085735 A1, which is hereby incorporated by reference in its entirety in the present disclosure. ST266 contains approximately 1700 unique molecules (FIG. 1). Accordingly, ST266* may contain at least approximately 1700 unique molecules, and the administration of ST266* at low volumes and concentrations may provide unexpected physiological responses even compared to ST266.

In preferred aspects, the ST266* secreted by the immortalized AMP cells of the present disclosure has higher levels of at least one or more of total protein, PDGF-BB, VEGF, TIMP1, TIMP2, Angiogenin, Schwann cell proliferation, and MMP9 inhibition compared to ST266. In other aspects, the ST266* secreted by the immortalized AMP cells of the present disclosure has lower levels of EGF compared to ST266. The ST266* obtained from the immortalized AMP cells of the present disclosure may have greater reduction in inflammatory cytokine levels compared to ST266 obtained from primary, non-immortalized, AMP cells.

The present disclosure provides methods of obtaining the secretome of the immortalized monoclonal AMP cells described herein, ST266*. The ST266* may be obtained from the secretions of the immortalized AMP cells, wherein a total protein concentration may be from 0.5 mg/mL to 6 mg/mL. According to certain aspects, the total protein concentration may be, without limitation, from 0.5 mg/mL to 1.0 mg/mL, 1.0 mg/mL to 1.5 mg/mL, 1.5 mg/mL to 2.0 mg/mL, 2.0 mg/mL to 2.5 mg/mL, 2.5 mg/mL to 3.0 mg/mL, 3.0 mg/mL to 3.5 mg/mL, 3.5 mg/mL to 4 mg/mL, from 3.5 mg/mL to 4.5 mg/mL, from 3.5 mg/mL to 5 mg/mL, and from 3.5 mg/mL to 5.5 mg/mL. In other aspects, the total protein concentration may be, without limitation, from 4.0 mg/mL to 4.2 mg/mL, from 4.2 mg/mL to 4.4 mg/mL, from 4.4 mg/mL to 4.6 mg/mL, from 4.6 mg/mL to 4.8 mg/mL, from 4.8 mg/mL to 5.0 mg/mL, from 5.0 mg/mL to 5.2 mg/mL, from 5.2 mg/mL to 5.4 mg/mL, from 5.4 mg/mL to 5.6 mg/mL, from 5.6 mg/mL to 5.8 mg/mL, and from 5.8 mg/mL to 6.0 mg/mL.

In other aspects, the total protein concentration is at least 0.01, 0.1, 0.5, 1.0, 1.5, 2.0, 2.5, 3.0, 3.5, 3.6, 3.7, 3.8, 3.9, 4.0, 4.1, 4.2, 4.3, 4.4, 4.5, 4.6, 4.7, 4.8, 4.9, 5.0, 5.1, 5.1, 5.2, 5.3, 5.4, 5.5, 5.6, 5.7, 5.8, 5.9, or 6.0 mg/mL. In other aspects, the total protein concentration is not less than 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, or 3.5 mg/mL. Any combination of the upper and lower ranges may be used, such as, 4.0 mg/mL to 4.5 mg/mL, 5.0 mg/mL to 5.5 mg/mL, 5.5 mg/mL to 6.0 mg/mL, and 5.1 mg/mL to 5.3 mg/mL.

The ST266* secreted by the immortalized AMP cells of the present disclosure may comprise Epidermal Growth Factor (EGF) from 0 pg/mL to 8200 pg/mL. According to certain aspects, EGF may be present, without limitation, in amounts from 0 pg/mL to 0.001 pg/mL, 0.001 pg/mL to 0.5 pg/mL, 0.5 pg/mL to 1 pgm/mL, 1 pg/mL to 5 pg/mL, 5 pg/mL to 10 pg/mL, 10 pg/mL to 25 pg/mL, 25 pg/mL to 50 pg/mL 50 pg/mL to 150 pg/mL, 50 pg/mL to 300 pg/mL, 50 pg/mL to 500 pg/mL, 50 pg/mL to 1000 pg/mL, 50 pg/mL to 2000 pg/mL, 50 pg/mL to 4000 pg/mL, 50 pg/mL to 6000 pg/mL, and 50 pg/mL to 8200 pg/mL. EGF may be present in amounts from 50 pg/mL to 140 pg/mL, including, without limitation, 50 pg/mL to 60 pg/mL, 60 pg/mL to 70 pg/mL, 70 pg/mL to 80 pg/mL, 80 pg/mL to 90 pg/mL, 90 pg/mL to 100 pg/mL, 100 pg/mL to 110 pg/mL, 110 pg/mL to 120 pg/mL, 120 pg/mL to 130 pg/mL, and 130 pg/mL to 140 pg/mL. In some aspects, the ST266* of the present disclosure may not include a quantifiable amount or a detectable amount of ST266* according to the lower limit of quantification (LLOQ) of quantitative methods known in the art, including, but not limited to, an Enzyme Linked Immunosorbent Assay (ELISA) such as an EGF Human ELISA kit (Invitrogen, CA, USA; product number KHG0061). ST266* may include a concentration of EGF less than the LLOQ, which is understood to be the lower limit at which an assay can provide quantitative results. As used herein, a “quantifiable amount” or “detectable amount” is understood to be an amount greater than or equal to the LLOQ.

The ST266* secreted by the immortalized AMP cells of the present disclosure may comprise PDGF-BB from 1 pg/mL to 90 pg/mL. According to certain aspects, PDGF-BB may be present, without limitation, in amounts from 1 pg/mL to 10 pg/mL, 1 pg/mL to 20 pg/mL, 1 pg/mL to 30 pg/mL, 1 pg/mL to 40 pg/mL, 1 pg/mL to 50 pg/mL, 1 pg/mL to 60 pg/mL, 1 pg/mL to 70 pg/mL, 1 pg/mL to 80 pg/mL, and 1 pg/mL to 90 pg/mL. In preferred aspects, PDGF-BB may be present in amounts from 10 pg/mL to 70 pg/mL, including, without limitation, 10 pg/mL to 20 pg/mL, 20 pg/mL to 30 pg/mL, 30 pg/mL to 40 pg/mL, 40 pg/mL to 50 pg/mL, 50 pg/mL to 60 pg/mL, and 60 pg/mL to 70 pg/mL.

The ST266* secreted by the immortalized AMP cells of the present disclosure may comprise vascular endothelial growth factor (VEGF) from 400 pg/mL to 7000 pg/mL. According to certain aspects, VEGF may be present, without limitation, in amounts from 400 pg/mL to 1000 pg/mL, 400 pg/mL to 2000 pg/mL, 400 pg/mL to 3000 pg/mL, 400 pg/mL to 4000 pg/mL, 400 pg/mL to 5000 pg/mL, 400 pg/mL to 6000 pg/mL, and 400 pg/mL to 7000 pg/mL. In preferred aspects, VEGF may be present in amounts from 2000 pg/mL to 7000 pg/mL, including, without limitation, 2000 pg/mL to 3000 pg/mL, 3000 pg/mL to 4000 pg/mL, 4000 pg/mL to 5000 pg/mL, 5000 pg/mL to 6000 pg/mL, and 6000 pg/mL to 7000 pg/mL,

The ST266* secreted by the immortalized AMP cells of the present disclosure may comprise TIMP metallopeptidase inhibitor 1 (TIMP1) from 1 ng/ml to 400 ng/mL. According to certain aspects, TIMP1 may be present, without limitation, in amounts from 1 ng/mL to 50 ng/mL, 1 ng/mL to 100 ng/mL, 1 ng/mL to 150 ng/mL, 1 ng/mL to 200 ng/mL, 1 ng/mL to 250 ng/ml, 1 ng/ml to 300 ng/mL, 1 ng/mL to 350 ng/mL, and 1 ng/mL to 400 ng/mL. In preferred aspects, TIMP1 may be present in amounts from 150 ng/ml to 375 ng/mL, including, without limitation, 150 ng/mL to 200 ng/mL, 200 ng/mL to 250 ng/ml, 250 ng/ml to 300 ng/mL, 300 ng/mL to 350 ng/mL, and 350 ng/ml to 375 ng/mL.

The ST266* secreted by the immortalized AMP cells of the present disclosure may comprise TIMP metallopeptidase inhibitor 2 (TIMP2) from 5 ng/ml to 500 ng/mL. According to certain aspects, TIMP2 may be present, without limitation, in amounts from 5 ng/mL to 100 ng/mL, 5 ng/mL to 200 ng/ml, 5 ng/mL to 300 ng/mL, 5 ng/mL to 400 ng/ml, and 5 ng/ml to 500 ng/mL. In preferred aspects, TIMP2 may be present in amounts from 250 ng/mL to 500 ng/mL, including, without limitation, 250 ng/ml to 300 ng/ml, 300 ng/ml to 350 ng/mL, 350 ng/mL to 400 ng/mL, 400 ng/mL to 450 ng/mL, and 450 ng/ml to 500 ng/mL.

The ST266* secreted by the immortalized AMP cells of the present disclosure may comprise Angiogenin from 100 pg/mL to 8000 pg/mL. According to certain aspects, Angiogenin may be present, without limitations, in amounts from 100 pg/mL to 1000 pg/mL, 100 pg/mL to 2000 pg/mL, 100 pg/mL to 3000 pg/mL, 100 pg/mL to 4000 pg/mL, 100 pg/mL to 5000 pg/mL, 100 pg/mL to 6000 pg/mL, 100 pg/mL to 7000 pg/mL, and 100 pg/mL to 8000 pg/mL. In preferred aspects, Angiogenin may be present in amounts of at least 2000 pg/mL, including, without limitation, 2000 pg/mL to 3000 pg/mL, 3000 pg/mL to 4000 pg/mL, 4000 pg/mL to 5000 pg/mL, 5000 pg/mL to 6000 pg/mL, 6000 pg/mL to 7000 pg/mL, and 7000 pg/mL to 8000 pg/mL.

The ST266* secreted by the immortalized AMP cells of the present disclosure may inhibit matrix metalloproteinase-9 (MMP9). The percentage of MMP9 inhibition may range from 4% to 90%. According to certain aspects, the percentage of MMP9 inhibition may be, without limitations, 4% to 10%, 4% to 20%, 4% to 30%, 4% to 40%, 4% to 50%, 4% to 60%, 4% to 70%, 4% to 80%, and 4% to 90%. In preferred aspects, the percentage of MMP9 inhibition may be 50 to 90%, including, without limitation, 50% to 60%, 60% to 70%, 70% to 80%, 80% to 90%, 60% to 65%, 60% to 70%, 60% to 80%, and 60% to 90%.

The ST266* secreted by the immortalized AMP cells of the present disclosure may have a Schwann Cell proliferation percentage of from 45% to 98%. According to certain aspects, the percentage of Schwann Cell proliferation may be, without limitations, 45% to 55%, 45% to 65%, 45% to 75%, 45% to 85%, 45% to 95%, and 45% to 98%. In preferred aspects, the Schwann Cell proliferation may be 70% to 95%, including, without limitation, 70% to 75%, 75% to 80%, 80% to 85%, 85% to 90%, and 90% to 95%.

According to some aspects, the ST266* of the present disclosure may include 0.5 mg/mL to 6 mg/mL total protein concentration, 0 pg/mL to 8200 pg/mL EGF, 1 pg/mL to 90 pg/mL PDGF-BB, 400 pg/mL to 7000 pg/mL VEGF, 1 ng/mL to 400 ng/ml TIMP1, 5 ng/mL to 500 ng/mL TIMP2, 100 pg/mL to 8000 pg/mL Angiogenin, a percentage of MMP9 inhibition of 4% to 90%, and a Schwann Cell proliferation percentage of 45% to 98%.

The present disclosure provides a method of manufacturing ST266* comprising, culturing at least one immortalized AMP cell of the present disclosure until they reach confluence. The culture medium may be STM100, alternative medium as described herein, or Iscove's Modified Dulbecco's Medium (IMDM) without phenol red and with addition of 5 mg/mL Human Serum Albumin (HSA). STM100 may be further supplemented with the recombinant human epidermal growth factor (rhEGF). While STM100, alternative medium, and IMDM are described herein, other medias are possible and within the scope of the present disclosure. One of ordinary skill in the art may select an appropriate media and appropriate concentrations of components added to the media according to the desired outcome of immortalization of AMP cells or generation of ST266*. While STM100 medium with HSA and EGF may result in a higher growth rate and ST266* production, the immortalized AMP cells of the present disclosure do not require HSA or EFG or any equivalent or similar component known in the art to proliferate, while non-immortalized AMP cells require HSA and EFG to proliferate. It has been shown that neither HSA nor STM100 exert any effects on inflammation, demyelination, and retinal ganglion cell loss (Khan, et. al., 2019). Proliferating immortalized AMP cells without HSA is beneficial, as it would decrease unnecessary protein concentration. Furthermore, in media comprising HSA, it is more difficult to remove proteins. Accordingly, proliferating immortalized AMP cells without HSA provides a more concentrated ST266* product, allowing for a smaller volume of ST266* necessary for therapeutic treatments and for a greater range of concentrations of ST266* components in dosing. HSA may also overwhelm analytical methods such as protein assays due to its protein concentrations. The secretome (ST266*) of the present disclosure may be free, substantially free, essentially free, or completely free of HSA. The secretome (ST266*) of the present disclosure may be free, substantially free, essentially free, or completely free of EGF.

Thus, the methods of the present disclosure include generating ST266* by culturing any of PB-E6-E7-PB-TERT, including, but not limited to, clones: PB-E6-E7-PB-TERT—clone 1, PB-E6-E7-PB-TERT—clone 2, PB-E6-E7-PB-TERT—clone 3, PB-E6-E7-PB-TERT—clone 4, PB-E6-E7-PB-TERT—clone 5, PB-E6-E7-PB-TERT—clone 6, PB-E6-E7-PB-TERT—clone 7, PB-E6-E7-PB-TERT—clone 8, PB-E6-E7-PB-TERT—clone 9, and PB-E6-E7-PB-TERT—clone 10 in STM100, alternative medium, or Iscove's Modified Dulbecco's Medium (IMDM) without phenol red and with addition of 5 mg/mL Human Serum Albumin (HSA) and/or 14 ng/ml human epidermal growth factor (rhEGF). While STM100, alternative medium, and IMDM are described herein, other medias are possible and within the scope of the present disclosure. While 5 mg/mL HSA and/or 14 ng/mL rhEGF are described herein, other concentrations and ranges are possible and within the scope of the present disclosure. One of ordinary skill in the art may select an appropriate media and appropriate concentrations of components added to the media according to the desired outcome of immortalization of AMP cells or generation of ST266*.

Methods of the present disclosure also include generating ST266* by culturing any of PB-E6-E7-PB-TERT, including, but not limited to, clones: PB-E6-E7-PB-TERT—clone 1, PB-E6-E7-PB-TERT—clone 2, PB-E6-E7-PB-TERT—clone 3, PB-E6-E7-PB-TERT-clone 4, PB-E6-E7-PB-TERT—clone 5, PB-E6-E7-PB-TERT—clone 6, PB-E6-E7-PB-TERT clone 7, PB-E6-E7-PB-TERT—clone 8, PB-E6-E7-PB-TERT—clone 9, and PB-E6-E7-PB-TERT—clone 10 in STM100, alternative medium, or Iscove's Modified Dulbecco's Medium (IMDM) without phenol red, without Human Serum Albumin (HSA), and without human epidermal growth factor (rhEGF).

Once the immortalized AMP cells have reached confluence, the media is changed to fresh culture media. The immortalized AMP cells are then cultured for at least a second time for four (4) days in the same fresh culture media. While a time period for culture of four (4) days has been described, more or less time for culture is possible and within the scope of the present disclosure. After four (4) days, the culture media containing the ST266* is collected and fresh culture media of the previous step is added. The process of collecting the culture media and replenishing with fresh culture media after four days may be repeated at least a second time. While the prior art non-immortalized AMP cells are limited in the number of passages, the immortalized AMP cells of the present disclosure may be capable of at least 12 passages. The culture medium containing the ST266* may then be filtered to further purify the composition, such as filtering the medium through a 0.22 μm pore size filter. The filtration step does not trap or capture proteins, as the proteins are still at physiological concentrations after the filtration step.

The present disclosure provides pharmaceutical compositions of ST266*, optionally in a pharmaceutically acceptable carrier. The term “pharmaceutically acceptable” means approved by a regulatory agency of the Federal or a state government of listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable, or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanal, and the like. The composition, if desired, can also contain minor amounts of wetting or emulsifying agents, or pH buffering agents. These compositions can take the form of solutions, suspensions, emulsions, tablets, pills, capsules, powders, sustained-release formulations, and the like.

The pharmaceutical compositions of the present disclosure may be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Compositions comprising ST266* may be formulated in any conventional manner optionally using one or more physiologically acceptable carriers and further optionally comprising excipients and auxiliaries. Proper formulation is dependent upon the route of administration chosen. The compositions may be packaged with written instructions for their use in treating, for example, wounds. The compositions may also be administered to the recipient in one or more physiologically acceptable carriers. Carriers for ST266* may include carriers suitable for sustained-release of ST266*. ST266* compositions of the present disclosure may be formulated for sustained-release, targeted-release, timed-release, extended-release, and the like, and may be used alone or in combination with an active or inactive agent. Skilled artisans are familiar with sustained-release/timed-release/targeted-release/extended-release formulation methodologies.

ST266* compositions of the present disclosure may be prepared in a variety of ways depending on the intended use of the compositions. For example, a composition useful in practicing the present disclosure may be a liquid including an agent of the present disclosure, such as ST266* in solution, in suspension, or both (solution/suspension). The term “solution/suspension” may refer to a liquid composition wherein the first portion of the active agent is present in solution and a second portion of the active agent is present in particulate form, in suspension in a liquid matrix. A liquid composition also includes a gel. The liquid composition may be aqueous or in the form of an ointment, salve, cream, or the like.

Compositions including ST266* may be cell free compositions, wherein “cell free composition” refers to a composition which does not contain intact cells.

ST266* compositions may be formulated in any conventional manner using one or more physiologically acceptable carriers. Proper formulation is dependent upon the route of administration chosen. For topical administration, the ST266* compositions may be formulated as a spray, liquid, cream, foam, gel, lotion, salve, ointment, etc. The composition may be administered as an oral liquid, a capsule, or a tablet designed to release ST266* compositions at a specific portion of the gastro-intestinal tract. For subcutaneous or intramuscular administration, the composition may be delivered by needle and syringe, by pen injectors, by needleless injection devices, and the like. For intranasal administration, the formulation may be administered as a nasal spray, a nebulized pulmonary dosage form, a metered dose inhaler, or a dry powder inhaler.

The present disclosure also provides for an article of manufacturing comprising packaging material and a pharmaceutical composition of the invention contained within the packaging material, wherein the pharmaceutical composition comprises compositions of ST266*. The packaging material comprises a label or package insert which indicates the use of the ST266*.

### Aspects of the Invention

The following aspects are disclosed in this application:

Aspect 1: An in vitro immortalized amnion-derived multipotent progenitor (AMP) cell.

Aspect 2: The in vitro immortalized AMP cell according to aspect 1, wherein the in vitro immortalized AMP cell is obtained by transferring hTERT gene located between a pair of site-specific recombination sequences into an amnion-derived multipotent progenitor cell.

Aspect 3: The in vitro immortalized AMP cell according to aspect 1, wherein the in vitro immortalized AMP cell is obtained by transferring HPV-16 E6/E7 located between a pair of site-specific recombination sequences into an amnion-derived multipotent progenitor cell.

Aspect 4: The in vitro immortalized AMP cell according to aspect 1, wherein the in vitro immortalized AMP cell is obtained by transferring HPV-16 E6/E7 and/or hTERT located between a pair of site-specific recombination sequences into an amnion-derived multipotent progenitor cell.

Aspect 5: The in vitro immortalized AMP cell according to any one of aspects 1 through 4, wherein the AMP cell is transfected with PiggyBac transposon stable construct encoding hTERT and/or HPV-16 E6/E7 under the control of human E2Fa promoter.

Aspect 6: The in vitro immortalized AMP cell according to aspect 5, wherein the PiggyBac transposon stable construct is not fused with a reporter tag.

Aspect 7: The in vitro immortalized AMP cell according to aspect 1, which is proliferated in serum-free medium.

Aspect 8: The in vitro immortalized AMP cell according to anyone of aspects 1 through 7, wherein the transfection is via liposomes prepared by combining a pair of site-specific recombination sequences with a cationic lipid.

Aspect 9: The in vitro immortalized AMP cell according to anyone of aspects 1 through 8, wherein the transfecting occurs using Lipofectamine™ 2000.

Aspect 10: The in vitro immortalized AMP cell according to anyone of aspects 1 through 7, wherein the transfecting occurs via microspheres.

Aspect 11: The in vitro immortalized AMP cell according to aspect 10, wherein the microspheres are prepared by combining a pair of site-specific recombination sequences with a 25% polymer solution (1:2 v/v) volumetric ratio, and incubating at 30 minutes at 70° C. followed by cooling to 23° C.

Aspect 12: The in vitro immortalized AMP cell according to aspect 11, wherein the 25% polymer solution comprises 12.5% PVP (40,000 Daltons) and 12.5% PEG in 1M sodium acetate at pH 5.5.

Aspect 13: An immortalized AMP cell line according to any one of aspects 1 through 12.

Aspect 14: A composition comprising a secretome (ST266*) of the immortalized AMP cell line of aspect 13.

Aspect 15: A cell culture comprising a plurality of the immortalized AMP cells according to any one of aspects 1 through 14.

Aspect 16: A method for the production of an immortalized amnion-derived multipotent progenitor (AMP) cell, the method comprising: providing an isolated AMP cell, immortalizing the isolated AMP cell, and generating at least one immortalized monoclonal AMP cell line.

Aspect 17: A method for the production of an immortalized AMP cell, the method comprising: isolating an AMP cell from a human placenta, immortalizing the isolated AMP cell, and generating at least one immortalized monoclonal AMP cell lines.

Aspect 18: The method according to aspect 16 or 17, wherein the isolated AMP cell is transfected with HPV-16 E6/E7.

Aspect 19: The method according to aspect 16 or 17, wherein the isolated AMP cell is transfected with hTERT.

Aspect 20: The method according to aspect 16 through 19, wherein the isolated AMP cell is transfected with a combination of hTERT and HPV-16 E6/E7.

Aspect 21: The method according to aspect 12 or 13, wherein the isolated AMP cell is transfected with hTERT and/or HPV-16 E6/E7 via a PiggyBac transposon stable construct having an Ef1a promoter.

Aspect 22: The method according to anyone of aspects 16 through 21, wherein the transfection is via liposomes prepared by combining a pair of site-specific recombination sequences with a cationic lipid.

Aspect 23: The method according to anyone of aspects 16 through 22, wherein the transfecting occurs using Lipofectamine™ 2000.

Aspect 24: The method according to anyone of aspects 16 to 21, wherein the transfecting occurs via microspheres.

Aspect 25: The in method according to aspect 24, wherein the microspheres are prepared by combining a pair of site-specific recombination sequences with a 25% polymer solution (1:2 v/v) volumetric ratio, and incubating at 30 minutes at 70° C. followed by cooling to 23° C.

Aspect 26: The method according to aspect 25, wherein the 25% polymer solution comprises 12.5% PVP (40,000 Daltons) and 12.5% PEG in 1M sodium acetate at pH 5.5.

Aspect 27: The method according to any one of aspects 16 through 26, wherein the immortalized AMP cell secretes ST266*.

Aspect 28: A method of treating a disease or disorder, wherein the method comprises: administering a secretome to an individual, wherein the secretome is produced according to any one of aspects 16 to 27.

Aspect 29: A method of making an ST266* composition comprising: culturing an immortalized AMP cell according to anyone of aspects 1 to 13, changing the culture medium after four days, culturing the immortalized AMP cell a second time, collecting the culture medium comprising ST266* after four days, and replenishing the culture medium.

Aspect 30: The method of aspect 29, wherein the culture medium is collected and replenished every four days for at least 12 passages.

Aspect 31: The method of aspect 29 through 30, wherein the immortalized AMP cells proliferate in serum-free culture medium.

Aspect 32: The method according to anyone of aspects 29 through 31, wherein the culture medium comprising ST266* is filtered to purify the ST266*.

Aspect 33: An ST266* composition made by the method of aspects 29 through 32.

Aspect 34: The composition of aspect 33, which is a pharmaceutical composition.

Aspect 35: A kit comprising the pharmaceutical composition of aspect 34.

Aspect 36: A therapeutic component comprising the pharmaceutical composition of aspect 34.

Aspect 37: The therapeutic component of Aspect 36, suitable for treating a disease or disorder, wherein the method comprises: administering a secretome to an individual.

Aspect 38: The immortalized monoclonal AMP cell line according to any of the foregoing aspects, wherein the immortalized monoclonal AMP cell line originates from a single transfected AMP cell, giving rise to at least one identical cell, wherein the at least one identical cell is a clone of the single transfected AMP cell expressing PB-E6-E7-PB-TERT, and wherein each single transfected AMP cell generates an identical clone.

Aspect 39: The immortalized AMP cell line according to any of the foregoing aspects, wherein the immortalized AMP cell line is: PB-E6-E7-PB-TERT—clone 1.

Aspect 40: The immortalized AMP cell line according to any of the foregoing aspects, wherein the immortalized AMP cell line is: PB-E6-E7-PB-TERT—clone 2.

Aspect 41: The immortalized AMP cell line according to any of the foregoing aspects, wherein the immortalized AMP cell line is: PB-E6-E7-PB-TERT—clone 3.

Aspect 42: The immortalized AMP cell line according to any of the foregoing aspects, wherein the immortalized AMP cell line is: PB-E6-E7-PB-TERT—clone 4.

Aspect 43: The immortalized AMP cell line according to any of the foregoing aspects, wherein the immortalized AMP cell line is: PB-E6-E7-PB-TERT—clone 5.

Aspect 44: The immortalized AMP cell line according to any of the foregoing aspects, wherein the immortalized AMP cell line is: PB-E6-E7-PB-TERT—clone 6.

Aspect 45: The immortalized AMP cell line according to any of the foregoing aspects, wherein the immortalized AMP cell line is: PB-E6-E7-PB-TERT—clone 7.

Aspect 46: The immortalized AMP cell line according to any of the foregoing aspects, wherein the immortalized AMP cell line is: PB-E6-E7-PB-TERT—clone 8.

Aspect 47: The immortalized AMP cell line according to any of the foregoing aspects, wherein the immortalized AMP cell line is: PB-E6-E7-PB-TERT—clone 9.

Aspect 48: The immortalized AMP cell line according to any of the foregoing aspects, wherein the immortalized AMP cell line is: PB-E6-E7-PB-TERT—clone 10.

Aspect 49: The immortalized AMP cell according to any of the foregoing aspects, wherein the ST266* composition comprises 0.5 mg/mL to 6 mg/mL total protein concentration, 0 pg/mL to 8200 pg/mL EGF, 1 pg/mL to 90 pg/mL PDGF-BB, 400 pg/mL to 7000 pg/mL VEGF, 1 ng/mL to 400 ng/mL TIMP1, 5 ng/ml to 500 ng/ml TIMP2, and 100 pg/mL to 8000 pg/mL Angiogenin.

Aspect 50: The immortalized AMP cell according to any of the foregoing aspects, wherein the ST266* composition has a percentage of MMP9 inhibition of 4% to 90%.

Aspect 51: The immortalized AMP cell according to any of the foregoing aspects, wherein the ST266* composition has a Schwann Cell proliferation percentage of 45% to 98%.

Aspect 52: The immortalized AMP cell according to any of the foregoing aspects, wherein the ST266* composition does not comprise a detectable amount of EGF.

Aspect 53: The ST266* composition according to any of the foregoing aspects, wherein the ST266* composition comprises 0.5 mg/mL to 6 mg/mL total protein concentration, 0 pg/mL to 8200 pg/mL EGF, 1 pg/mL to 90 pg/mL PDGF-BB, 400 pg/mL to 7000 pg/mL VEGF, 1 ng/mL to 400 ng/mL TIMP1, 5 ng/mL to 500 ng/mL TIMP2, and 100 pg/mL to 8000 pg/mL Angiogenin.

Aspect 54: The ST266* composition according to any of the foregoing aspects, wherein the ST266* composition does not comprise a detectable amount of EGF.

Aspect 55: The method for the production of an immortalized AMP cell according to any of the foregoing aspects, wherein the culture medium is free, substantially free, essentially free, or completely free of HSA.

Aspect 56: The method for the production of an immortalized AMP cell according to any of the foregoing aspects, wherein the culture medium is free, substantially free, essentially free, or completely free of EGF.

Aspect 57: The method for the production of an immortalized AMP cell according to any of the foregoing aspects, wherein the culture medium is free, substantially free, essentially free, or completely free of EGF and HSA.

Aspect 58: The immortalized AMP cell according to any of the foregoing aspects, wherein the ST266* composition is free, substantially free, essentially free, or completely free of HSA.

Aspect 59: The immortalized AMP cell according to any of the foregoing aspects, wherein the ST266* composition is free, substantially free, essentially free, or completely free of EGF.

Aspect 60: The immortalized AMP cell according to any of the foregoing aspects, wherein the ST266* composition is free, substantially free, essentially free, or completely free of EGF and HSA.

Aspect 61: The ST266* composition according to any of the foregoing aspects, wherein the ST266* composition is free, substantially free, essentially free, or completely free of EGF.

Aspect 62: The ST266* composition according to any of the foregoing aspects, wherein the ST266* composition is free, substantially free, essentially free, or completely free of HSA.

Aspect 63: The ST266* composition according to any of the foregoing aspects, wherein the ST266* composition is free, substantially free, essentially free, or completely free of EGF and HSA.

### EXAMPLES

### Example 1: Immortalization of AMP Cells and Generation of Monoclonal Cell Lines

### AMP Cell Culture

Amnion derived cells were obtained from donated human full-term placentas as described in R. Banas 2008. Cells were banked by cryopreservation in CryoStor™ CS-5 (BioLife Solutions, Bothell, WA, USA) and stored in the vapor phase of liquid nitrogen. The cells were subsequently thawed and cultured in T-75 tissue culture flasks in proprietary serum-free culture medium STM 100 (Irvine Scientific Santa Ana, CA, USA) with addition of recombinant human epidermal growth factor (rhEGF, 14 ng/mL) (R&D Systems, Minneapolis, MN, USA) at cell density 6×10⁵ per cm² at 37° C., 5% carbon dioxide, and 95% air humidity. At sub-confluency, the cell culture medium was removed, and cells were washed with Hanks' Balanced Salt Solution (HBSS) (Thermo Fisher Scientific, Waltham, MA, USA) to remove any unattached cells. Adherent cells were then enzymatically detached and plated into wells of 12 well plates for transfections.

### Transfection

Primary AMP cells were seeded into individual wells of 12 well plates at the density of 4×10⁵ per cm² in STM100 medium supplemented with rhEGF, 14 ng/mL. The next day, or at 80% confluency, cells were transfected with hTERT alone, HPV-16 E6/E7 alone, or hTERT and HPV-16 E6/E7 in combination. Negative control was represented by un-transfected cells and positive controls were cells transfected with GFP encoding construct. Transfections were performed using Lipofectamine™ 2000 Transfection Reagent (Thermo Fisher Scientific, Waltham, MA, USA) according to manufacturer protocol or using microspheres (U.S. Pat. No. 9,339,465 (Brown et al.)).

Microspheres were prepared by combining DNA (1-1.5 μg/cm², transposase/transposon ratio of 1:2) with human serum albumin (HSA) (Octapharma Plasma, Inc., Charlotte, North Carolina, USA). HAS was dissolved in acetate buffer, 1M, pH 5.5 to the final concentration of 10 mg/mL (1%). Poly-L-Lysine.Hbr (PLL) (MP Biomedicals, 300 Industry Drive, Pittsburgh, USA) was added to the DNA solution in a ratio of 1:1. A 25% polymer solution was composed of 12.5% PVP (40,000 Daltons) and 12.5% PEG in 1M sodium acetate at pH 5.5. The polymer solution was added to the DNA-PLL mixture in a 2:1 volumetric ratio. The final ratios of DNA-HSA:PLL:25% PEG/PVP were 1:1:2. Specifically, the DNA-HSA solution was 750 μL, PLL was 750 μL, and 25% PEG/PVP was 3000 μL. The mixed solutions were incubated for 30 minutes at 70° C. and then cooled to 23° C. The cooled solutions then became turbid with visible precipitation. The suspensions were then centrifuged, excess PEG/PVP was removed, and the resulting pellets were washed three times by resuspending deionized water and centrifugation. Pellets were finally resuspended in the fresh cell culture medium and added to the cells. The culture medium was replaced the next day.

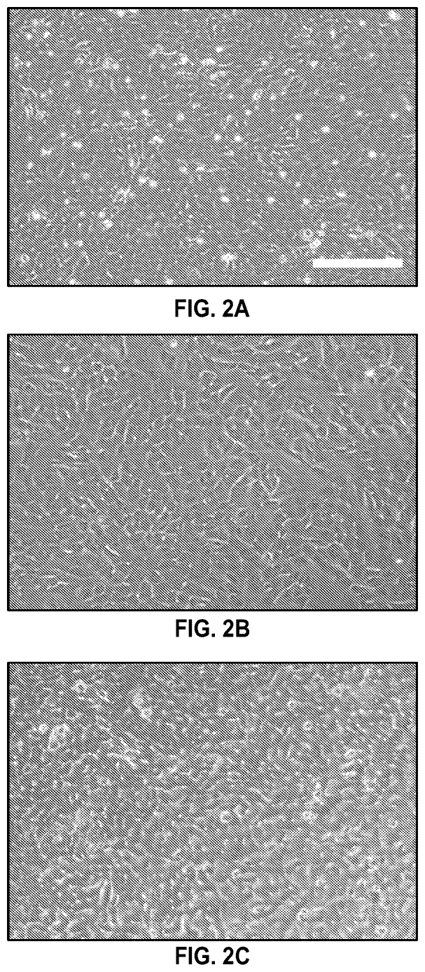

Cells transfected with microspheres appeared healthier and had higher proliferation rates immediately after transfection compared to cells transfected with Lipofectamine™ 2000 Transfection Reagent. The morphology of transfected cells was consistent with the primary AMP cells (FIGS. 2A through 2C).

### Constructs

The E6/E7 region of HPV-16 and the catalytic subunit of hTERT were inserted into a piggyBAC transposon stable vector with Ef1a promoter (VectorBuilder Inc., Chicago, IL, USA). Transposase was encoded in the Hyperactive piggyBac transposase mammalian expression vector (VectorBuilder Inc., Chicago, IL, USA).

### Generation of Immortalized Monoclonal Cell Lines

Transfected AMP cells were seeded onto 15 cm plates in density of 100, 200, and 400 cells per plate. Attached single cells were identified and marked on the bottom of the plate. Colonies originating from the single cells were them removed using sterile cloning disks (SP Bel-Art, Wayne, NJ, USA) and transferred into wells of 24 well plates. Upon sub-confluency, cells were replated into 3 cm, 6 cm and later 10 cm plates for further expansion and characterization.

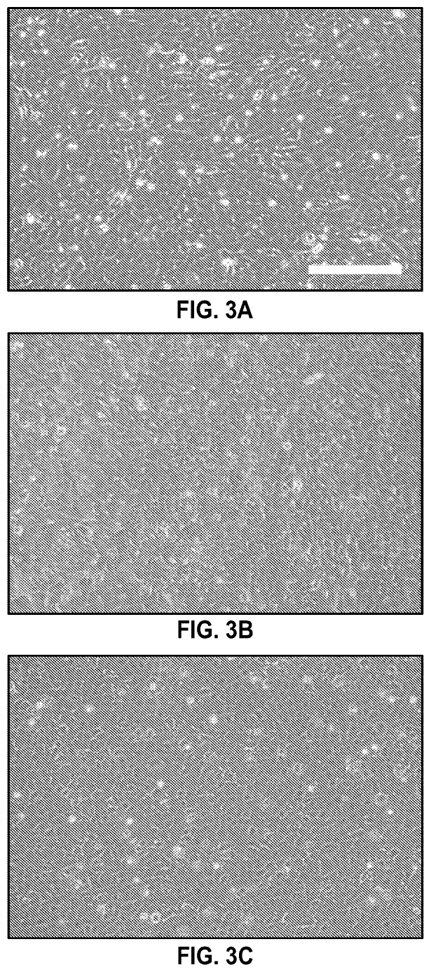

The immortalized cell lines actively proliferated and exhibited and exponential increase in cell density. After 6 months in culture, the proliferation of cells transfected with only HPV-16 E6/E7 or only hTERT decreased. Only cell lines co-transfected with both HPV-16 E6/E7 and hTERT in combination demonstrated continuous growth acceleration with prolonged time in culture. Thus, the co-transfected cells were used for further characterization. The co-transfected cell lines exhibited accelerated growth and a cobblestone-like epithelial morphology (FIGS. 3A through 3C)

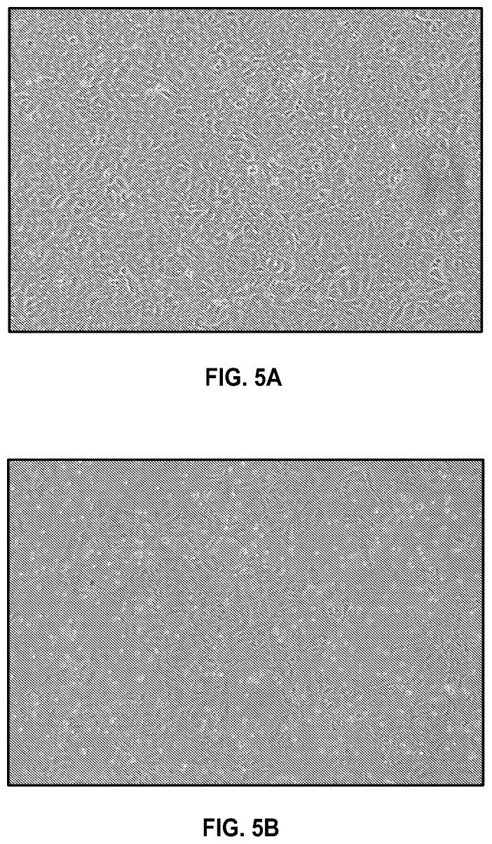

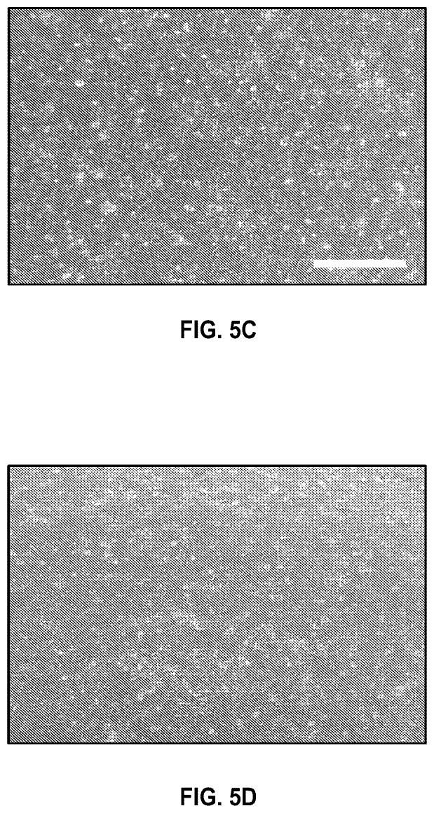

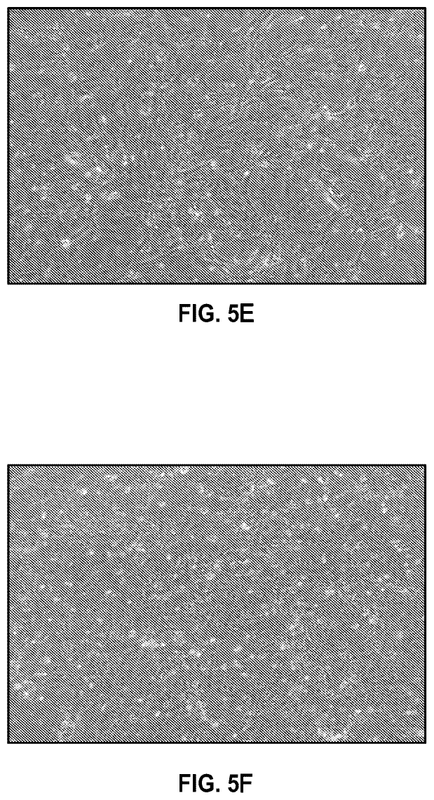

The immortalized AMP cell lines, just like primary AMP cells, were cultured in the serum free medium with addition of rhEGF. The immortalized AMP cells, unlike the primary AMP cells (FIGS. 5A & 5B), grew and proliferated in the same serum free medium without rhEGF, and the removal of rhEGF did not affect growth or morphology (FIGS. 5C and 5D). Furthermore, immortalized cells could be cultured and passaged in REM medium, in absence of both HSA and EGF (FIGS. 5E & 5F)

### PCR

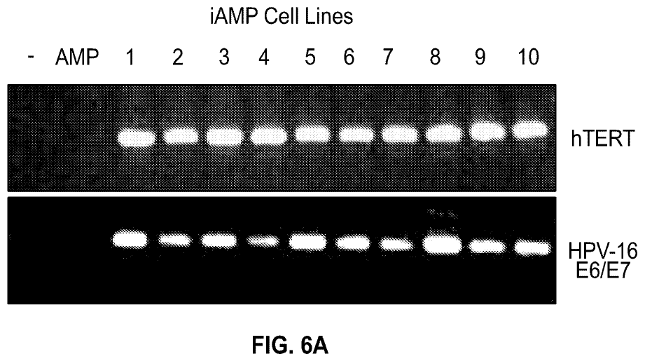

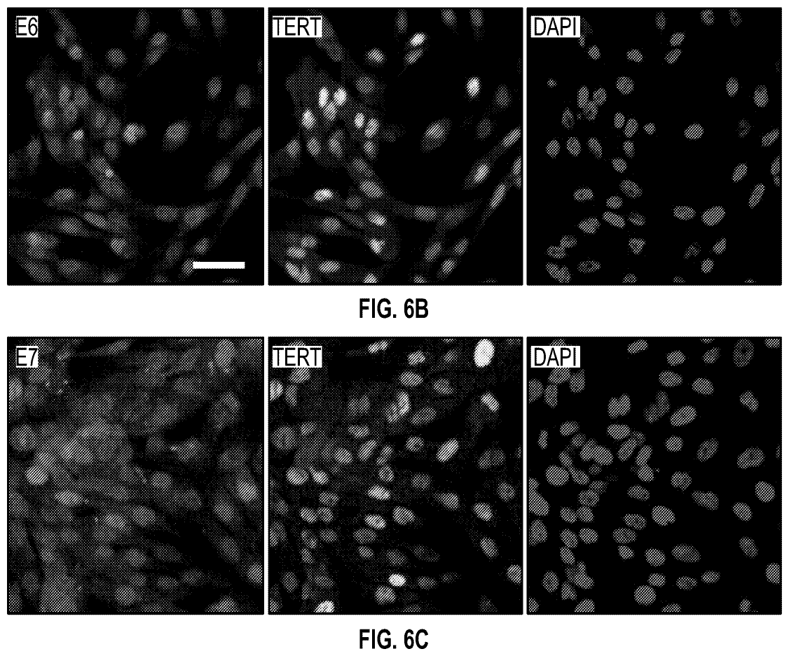

Genomic DNA from the immortalized AMP cells was extracted with PureLink™ Genomic DNA Mini Kit (Invitrogen, Waltham, MA, USA). PCR was performed using Platinum Taq DNA Polymerase High Fidelity (Thermo Fisher Scientific, Waltham, MA, USA) to amplify hTERT and HPV16-E6/E7 transgenes. PCR products were separated by 2% agarose gel electrophoresis at 90V for 40 minutes. The gels were stained with ethidium bromide and the correct size fragments were confirmed by imaging using UVP BioImaging Systems and purified from agarose gels using the QIAquick Gel Extraction Kit (Quiagen, Germantown, MD, USA) (FIG. 6A). The correct sequences were then confirmed by sequencing (Azenta, Waltham, MA, USA).

### Immunocytochemistry

The insertion of HPV-16 E6/E7 and hTERT was verified by sequencing and immunocytochemistry to determine whether combined activity of overexpressed proteins is responsible for immortalization of AMP cells. Immortalized cell lines were analyzed by immunocytochemistry for expression of HPV E6 and E7 and hTERT.

Images (FIG. 6B) demonstrated strong nuclear expression of hTERT and the presence of both E6 and E7 proteins in all immortalized AMP cells. The E6 protein was localized in the nuclei of the cells, and the E7 protein was visible mostly in the nuclei but also in the cytoplasm. E7 proteins are known to localize in the nuclei and cytoplasm in subconfluent cells but become cytoplasmic in confluent cells. The immortalized AMP cells were grown to sub-confluency before each passaging, thus, both nuclear and cytoplasmic localization was observed.

Cells were washed with Hanks' balanced salt solution and fixed in 4% paraformaldehyde for 18 minutes. Fixed cells were rinsed 3 times with PBS, permeabilized with 0.5% Triton 100× in PBS for seven minutes and then blocked with 1% bovine serum albumin (BSA) for one hour. Incubation with the primary antibody took place overnight at 4° C. Primary antibodies used were Anti-HPV16 E6/18 E6 Antibody (C1P5, 1:25), Anti-HPV16 E7 Antibody (ED17, 1:25) and Anti-TERT Antibody (A-6, 1:25) (Santa Cruz Biotechnology Inc., Dallas, TX, USA). After overnight incubation with primary antibodies, cells were rinsed three times with PBS and incubated with m-IgG1 BP-CFL 488 or m-IgG1 BP-CFL 594 secondary antibodies (Santa Cruz Biotechnology Inc., Dallas, TX, USA) diluted in PBS for 2 hours at room temperature. Then, the secondary antibody solution was removed, and the cells were rinsed 3 times using PBS. Coverslips were mounted on glass slides under UltraCruz® Aqueous Mounting Medium with DAPI (Santa Cruz Biotechnology Inc., Dallas, TX, USA). Immunoreactivity was observed and analyzed using Zeiss LSM 880 confocal laser scanner microscope. Controls for aberrant primary antibody cross-reactivity

The phenotype of the cells was observed by microscopy and surface marker expression by flow cytometry analysis (CD45, CD49, CD104, CD140, HLAG, SSEA4, PD-L2 PE, HLAG-PE).

Primary AMP cells, immortalized polyclonal cells, and immortalized cell lines 1 (PB-E6-E7-PB-TERT—clone 1), 8 (PB-E6-E7-PB-TERT—clone 8), and 9 (PB-E6-E7-PB-TERT—clone 9) were grown and passaged in culture. The cell growth curves were established by evaluating the population of doubling level (PDL) and plotted against time in culture. Growth curves of un-transfected primary AMP cells and immortalized polyclonal AMP culture (FIG. 4A) and growth curves of three monoclonal cell lines and PB-E6-E7-PB-TERT clones 1, 8, and 9 originating from the polyclonal AMP culture (FIG. 4B) were plotted.

The growth curves demonstrated that the immortalized polyclonal cells and monoclonal cell lines exhibited increasingly faster proliferation rates, while primary AMP cells exhibited only limited proliferation in culture.

### Example 2: Characterization of Secretome Composition

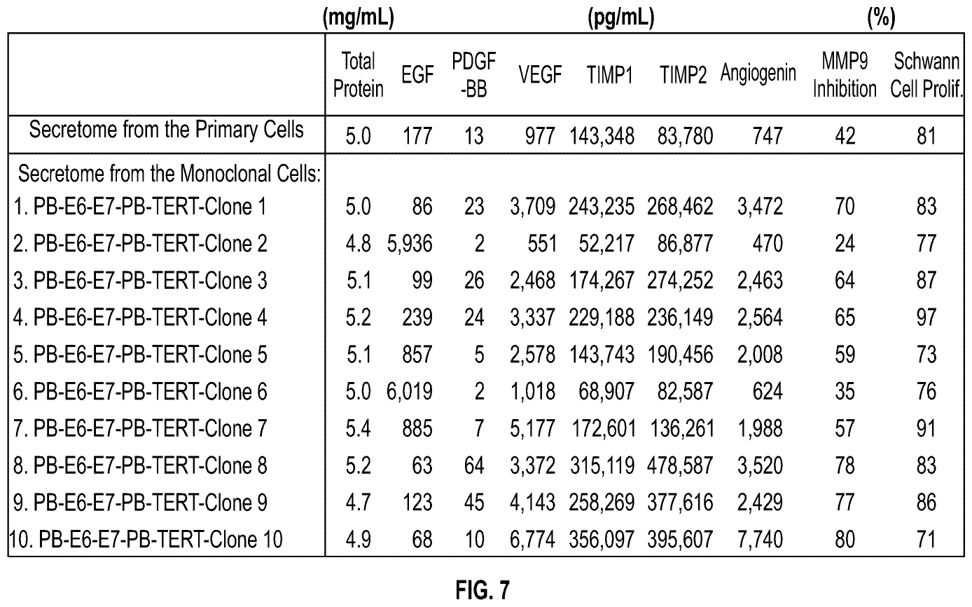

The secretome collected from immortalized monoclonal cell lines 1, 8, and 9 of FIG. 7, upon sub-confluency, was filtered through 0.22 μm pore size filter and analyzed for protein composition and functional properties by enzyme-linked immunosorbent assay (ELISA) for detection and quantification of Total Protein, PDGF-BB, VEGF, EGF, Angiogenin, TIMP-1, TIMP-2 and MMP9 inhibition. Schwann cells in vitro proliferation assay was performed based on kit manufacturer protocol. SW10 Mouse Schwann Cells (Cat #CRL-2766; ATCC, Manassas, VA) were seeded in 96-well tissue culture treated plates and cultured in normal growth media (NGM), containing Dulbecco's Modified Eagle Medium (DMEM; Cat #35050061) and 10% FBS (HyClone Cat #SH30071.01, GE Healthcare, Chicago, IL, USA), for a fixed amount of time at 37° C. and 5% carbon dioxide. When acclimated, NGM media was replaced by minimal starvation medium for 24 hours and subsequently replaced the secretomes from the immortalized cells. After 24 hours, proliferation of Schwann cells was determined using a commercially viable cell counting kit (Cat #96992, Milipore sigma, St. Louis, MO). All experiments were repeated three times.

The filtered secretome is referred to as ST266*.

The medium was STM100 supplemented with the recombinant human epidermal growth factor rhEGF (14 ng/ml).

The ST266* generated by each clone was analyzed for expression of PDGF-BB, VEGF, EGF, TIMP-1, ANGIOGENIN, TIMP2, DECORIN, CA-125, MMP9 inhibition, and Schwann cell proliferation. ST266* in terms of levels of total protein, EGF, PDGF-BB, VEGF, TIMP1, TIMP2, Angiogenin, MMP9 inhibition, protein aggregation, and Schwann cell proliferation was compared to an ST266 control generated from primary AMP cells (see FIG. 7).

The ST266* generated by each clone in a feeder-free culture system of STM100 in terms of levels of total protein, EGF, PDGF-BB, VEGF, TIMP1, TIMP2, Angiogenin, MMP9 inhibition, protein aggregation, and Schwann cell proliferation was compared to an ST266 control (FIG. 7). The ST266* exhibited comparable or higher values of therapeutic factors and functional potential as shown in FIG. 7.

### Example 3: Proinflammatory Cytokine Analysis in the Lipopolysaccharides (LPS) Mouse Model after Treatment with Secretome from Immortalized AMP Monoclonal Cell Lines

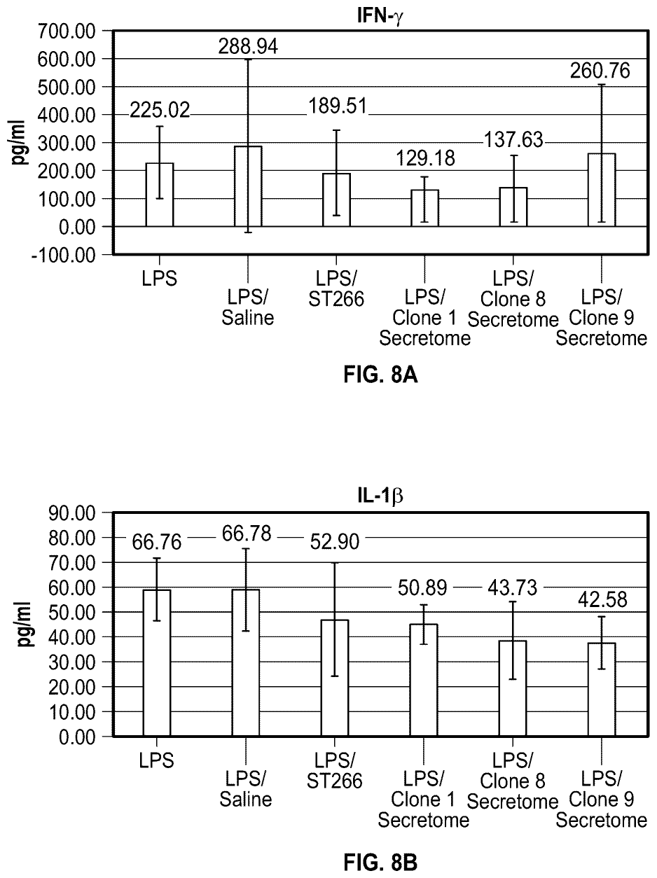

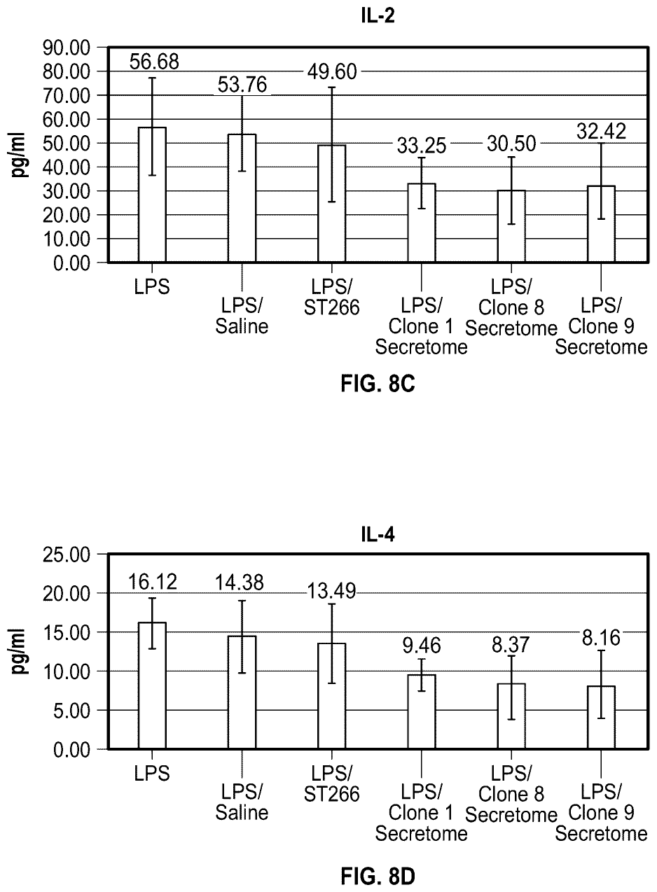

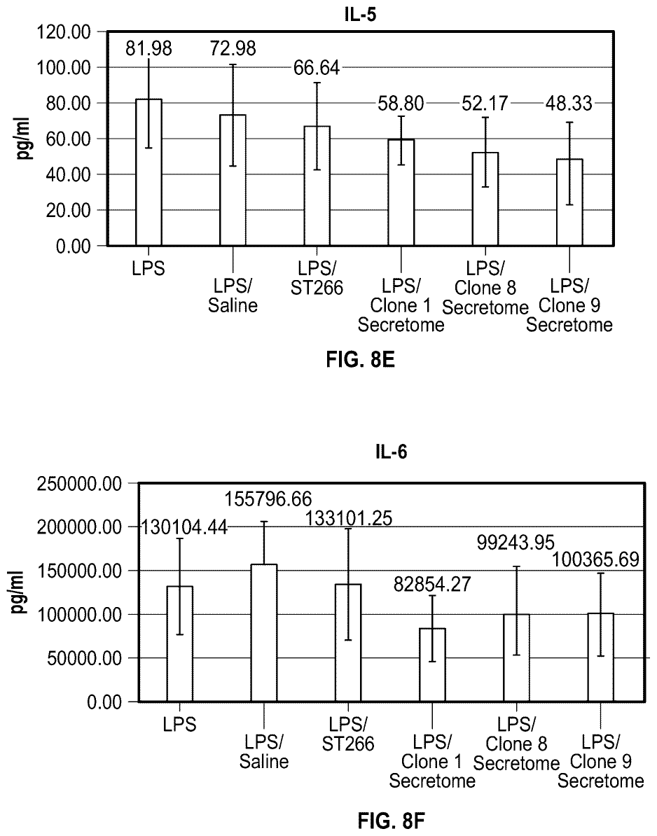

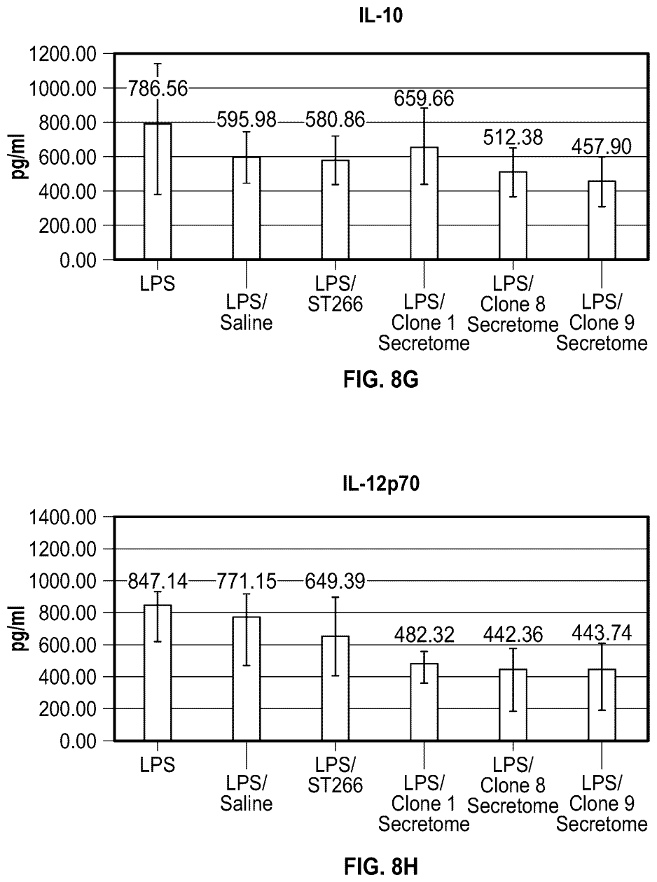

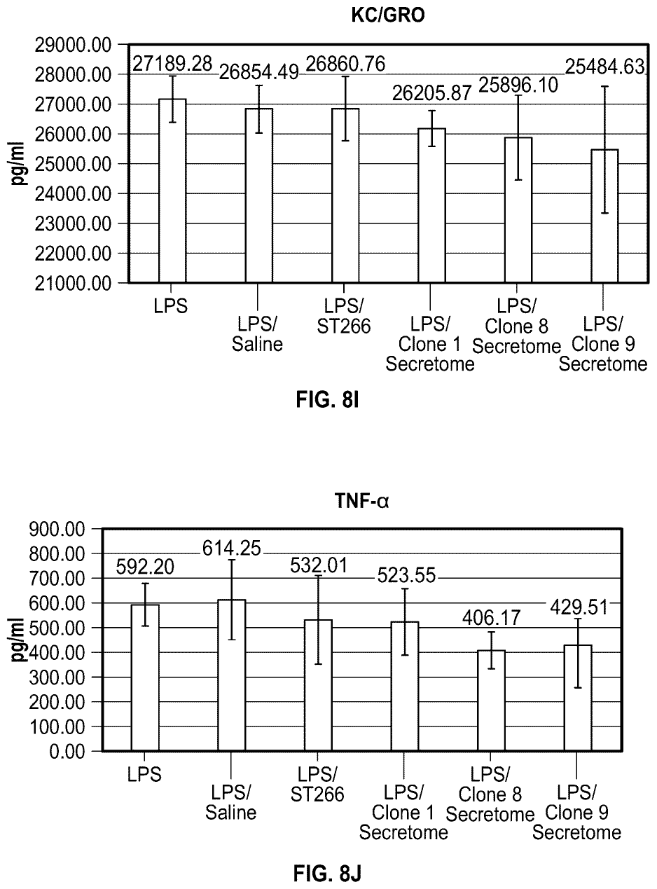

The secretome (ST266*) collected from immortalized monoclonal cell lines 1, 8, and 9 of FIG. 7 was filtered through a 0.22 μm filter. The mice were then treated with LPS (5 mg/kg) to one side of the abdomen and the secretome (ST266*) from the immortalized cell lines, primary cells (ST266), or saline (8 mL/kg) were administered immediately following to the opposite side of the abdomen by intraperitoneal injection. Serum was collected at 6 hours and quantitative measurements for ten biomarkers (interferon γ (IFN-γ) (FIG. 8A), interleukin-1β (IL-1β) (FIG. 8B), interleukin-2 (IL-2) (FIG. 8C), interleukin-4 (IL-4) (FIG. 8D), interleukin-5 (IL-5) (FIG. 8E), interleukin-6 (IL-6) (FIG. 8F), interleukin-10 (IL-10) (FIG. 8G), interleukin-12p70 (IL-12p70) (FIG. 8H), chemokine keratinocyte chemoattractant (KC)/human growth-regulated oncogene (KC/GRO) (FIG. 8I) and tumor necrosis factor a (TNF-α) (FIG. 8J)) that are associated with the inflammatory response and immune system regulation were performed by MSD Cytokine Assay.

LPS is endotoxin from the outer membrane of gram-negative bacteria. When injected into the intraperitoneal cavity of mice, high levels of pro-inflammatory cytokines are released. These cytokines can then be measured in circulating serum and lead to the development of systemic inflammatory response syndrome (SIRS) and eventually dose-dependent mortality (DG. Remick et al., 2000). The ST266* from three immortalized cell lines was examined for their ability to suppress the cytokine levels after LPS injection.

Results were analyzed by One Way Anova Statistics where p<0.05 was considered significant.

The results (FIGS. 8A through 8J) indicated that the ST266* from immortalized cell lines significantly reduced all inflammatory cytokine levels compared to the LPS/Saline group. The ability to suppress inflammation in vivo indicated that ST266* has strong anti-inflammatory therapeutic potential.

### Example 4: Immortalized AMP Cells do not Require HSA and EGF

Controls include cultured non-immortalized AMP cells in the presence of STM100 with HSA and 25 ng/mL rhEGF. Another control included non-immortalized AMP cells cultured in Iscove's Modified Dulbecco's Medium (IMDM) without HSA and without rhEGF. All flasks were harvested on day 21 for a total cell count.

Non-immortalized AMP cells with HSA and EGF included a total viable cell count of 3.01×10⁶ and non-immortalized AMP cells without HSA and without EGF had a total viable cell count of 2.52×10⁵.

AMP cells cultured in the presence of HSA and EGF resulted in significantly greater viable cell numbers compared to those cultured without HSA and EGF. As shown in FIG. 5A, non-immortalized AMP cells cultured with HSA and EGF are confluent at 10× magnification. FIG. 5B demonstrates that non-immortalized AMP cells without HSA and without EGF are dispersed and non-confluent at 10× magnification.

Immortalized AMP cells of the present disclosure do not require EGF and HSA in the culture medium to proliferate. FIG. 5C demonstrates a phase contrast image of PB-E6-E7-PB-TERT—clone 1 at 235 days in culture, grown and passaged 30 days without EGF. FIG. 5D demonstrates a phase contrast image of PB-E6-E7-PB-TERT—clone 8 at 235 days in culture, grown and passaged 30 days without EGF. FIG. 5E demonstrates a phase contrast image of PB-E6-E7-PB-TERT—clone 1 in alternative medium at 235 days in culture, grown and passaged in the presence of HSA and EGF for 30 days. FIG. 5F demonstrates a phase contrast image of PB-E6-E7-PB-TERT—clone 1 in alternative medium at 235 days in culture, grown and passaged without HSA and EGF for 30 days. FIGS. 5A-5F demonstrate that immortalized AMP cells, unlike non-immortalized AMP cells, can grow, proliferate, and be passaged without EGF and HSA.

The secretome (ST266) of non-immortalized AMP cells cultured with HSA and EGF and non-immortalized AMP cells cultured without HSA and EGF and the secretome (ST266*) of immortalized AMP cell PB-E6-E7-PB-TERT—clone 8 cultured without HSA and EGF were analyzed for expression of cytokine using an ELISA test. Cytokine concentration (pg/mL) and percent control of cytokine expression are shown in Tables 1 and 2, respectively.

TABLE 1
Cytokine Concentration (pg/mL)

| | PDGF-BB | VEGF | EGF | Angiogenin | TIMP1 | TIMP2 |
|---|---|---|---|---|---|---|
| Non-immortalized AMP cell | 45 | 4,771 | 65 | 680 | 453,500 | 304,295 |
| with HSA + EGF | | | | | | |
| PB-E6-E7-PB-TERT - clone 8 | 64 | 3372 | 63 | 3520 | 356,097 | 478,587 |
| Non-immortalized AMP cell | 58 | 1,856 | 15 | 422 | 66,418 | 168,234 |
| without HSA and EGF | | | | | | |

TABLE 2
Percent Control Cytokine Expression

| | PDGF-BB | VEGF | EGF | Angiogenin | TIMP1 | TIMP2 |
|---|---|---|---|---|---|---|
| Non-immortalized AMP cell | 100.00% | 100.00% | 100.00% | 100.00% | 100.00% | 100.00% |
| with HSA + EGF (Control) | | | | | | |
| PB-E6-E7-PB-TERT - clone 8 | 143.19% | 70.68% | 96.99% | 517.65% | 78.52% | 157.28% |
| Non-immortalized AMP cell | 128.78% | 38.91% | 23.23% | 62.08% | 14.65% | 55.29% |
| without HSA and EGF | | | | | | |

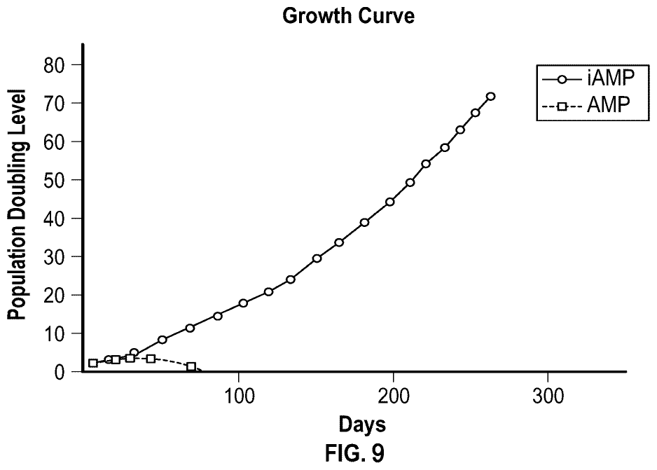

FIG. 9 demonstrates the cell population doubling level of immortalized AMP cells compared to non-immortalized AMP cells. FIG. 9 indicates that immortalized AMP cells continue to grow for at least 250 days compared to non-immortalized AMP cells which only survive less than 70 days.

### Example 5: ST266* Obtained from Immortalized AMP Cells Cultured without EGF and without Both EGF and HSA do not have Detectable Amounts of EGF

An immortalized AMP cell line (PB-E6-E7-PB-TERT-Cherry-polycloanal-d30) was cultured in STM100 with HSA and without EGF in STM100 medium. A second immortalized AMP cell line (PB-E6-E7-PB-TERT-Chearry-clone3-d93) was cultured in STM100 without both HSA and EGF. The secretome (ST266*) was collected according to the methods of the present disclosure, and an ELISA was performed to determine the composition of the secretome as shown in Table 3. As demonstrated by Table 3, the amount of EGF in the ST266* compositions collected was less than the lower limit of quantitation (LLOQ) of the assay.

TABLE 3
Secretome (ST266*) Composition

| Immortalized AMP cell | Total Protein | |
|---|---|---|
| line | (mg/mL) | EGF |
| PB-E6-E7-PB-TERT- | 4.4 | <LLOQ |
| Cherry-polycloanal-d30 | | |
| PB-E6-E7-PB-TERT- | 4.2 | <LLOQ |
| Chearry-clone3-d93 | | |

### REFERENCES

M. Toshio et al., Stem Cell Characteristics of Amniotic Epithelial Cells. Stem Cells; 23:1549-1559 (2005).
R. Weinberg, Bumps on the road to immortality. Nature 396, 23-24 (1998).
K. Zhou et al., Establishment and characterization of immortalized human amniotic epithelial cells. Cell Reprogram. 15 (1): 55-67 (2013)
T. Kiyono et al., Both Rb/p16INK4a inactivation and telomerase activity are required to immortalize human epithelial cells. Nature 396, 84-88 (1998)
H J. Muller, The remaking of chromosomes. Collecting Net. 13, 182-198 (1938)
B. McClintock, The origin and behavior of mutable loci in maize. Proc Natl Acad Sci USA 36(6): 344-355 (1950)
A G. Bodnar, et al., Extension of lifespan by introduction of telomerase into normal human cells. Science 279, 349-352 (1998)
T M. Nakamura et al., Reversing Time: Origin of Telomerase. Cell, Vol. 92, 587-590, (1998)
J W. Shay et al., Telomere shortening may contribute to aging and cancer: a perspective. Molecular and Cellular Differentiation, 2(1) 1-21, (1994)
R D. Ramirez, et al. Immortalization of human bronchial epithelial cells in the absence of viral oncoproteins. Cancer Research, (64) 9027-9034, (2004)
N. Dyson et al., The human papilloma virus-16 E7 oncoprotein is able to bind to the retinoblastoma gene product. Science, 243(4893) 934-7 (1989)
S. Cheng, et al., Differentiation-dependent up-regulation of the human papillomavirus E7 gene reactivates cellular DNA replication in suprabasal differentiated keratinocytes. Genes Dev, 9, 2335-2349 (1995)
J. Nevins, E2F: A link between the RB tumor suppressor protein and viral oncoproteins. Science, 258, 424-429 (1992)
E K. Yim et al., The Role of HPV E6 and E7 Oncoproteins in HPV-associated Cervical Carcinogenesis. Cancer Res Treat, 37(6) 319-324 (2005)
A. Kim, I. Pyykko, Size matters: versatile use of PiggyBac transposons as a genetic manipulation tool. Mol Cell Biochem 354, 301-309 (2011).
A. Lacoste et al., An Efficient and Reversible Transposable System for Gene Delivery and Lineage-Specific Differentiation in Human Embryonic Stem Cells. Cell Stem Cell 5, 332-342 (2009)
M H. Wilson et al., PiggyBac Transposon-mediated Gene Transfer in Human Cells. Molecular Therapy, (15) 139-145 (2007)
Q. Chen et al., Nature Communications volume 11, Article number: 3446 (2020)
L. Pray, Transposons: The jumping genes. Nature Education 1 (1): 204 (2008)
E R. Burnight, et al., A hyperactive transposase promotes persistent gene transfer of a piggyBac DNA transposon. Molecular Therapy-Nucleic Acids, e50 (2012)
K A. Skipper et al., DNA transposon-based gene vehicles-scenes from an evolutionary drive Journal of Biomedical Science 20:92 (2013)
S C Y. Wu et al., piggyBac is a flexible and highly active transposon as compared to Sleeping Beauty, Tol2, and Mos1 in mammalian cells. PNAS, 103 (41) 15008-15013, (2006)
Khan et al., Effects of Varying Intranasal Treatment Regimens in ST266-Mediated Retinal Ganglion Cell Neuroprotection. J Neuro-Opthalmol 39:191-199 (2019)

What is claimed is:

1. An immortalized amnion-derived multipotent progenitor (AMP) cell.

2. The immortalized AMP cell of claim 1, wherein the immortalized AMP cell is obtained by transferring an hTERT gene located between a pair of site-specific recombination sequences into a non-immortalized AMP cell.

3. The immortalized AMP cell of claim 1, wherein the immortalized AMP cell is obtained by transferring HPV-16 E6/E7 located between a pair of site-specific recombination sequences into non-immortalized AMP cell.

4. The immortalized AMP cell of claim 1, wherein the immortalized AMP cell is obtained by transferring HPV-16 E6/E7 and hTERT located between a pair of site-specific recombination sequences into a non-immortalized AMP cell.

5. The immortalized AMP cell of claim 1, wherein the immortalized AMP cell is transfected with a PiggyBac transposon stable construct encoding hTERT and HPV-16 E6/E7.

6. The immortalized AMP cell of any one of claims 1-5, wherein the immortalized AMP cell is proliferated in serum-free medium.

7. The immortalized AMP cell of claim 6, wherein the serum-free medium does not comprise human serum albumin (HSA).

8. The immortalized AMP cell of claim 6 or 7, wherein the serum-free medium does not comprise epithelial growth factor (EGF).

9. A composition comprising at least one immortalized AMP cell and a secretion of the at least one immortalized AMP cell.

10. The composition of claim 9, wherein the secretion is ST266*.

11. The composition of claim 10, wherein the ST266* does not comprise EGF.

12. A cell culture comprising a plurality of immortalized AMP cells.

13. A method for the production of an immortalized AMP cell, the method comprising:
providing an isolated non-immortalized AMP cell;
immortalizing the isolated non-immortalized AMP cell; and
generating at least one immortalized monoclonal AMP cell line.

14. The method of claim 13, wherein the method further comprises:
isolating the non-immortalized AMP cell from a human placenta.

15. The method of claim 13, wherein the method further comprises:
proliferating the at least one immortalized monoclonal AMP cell line in serum-free medium, wherein the serum-free medium does not comprise HSA.

16. The method of claim 13 or 15, wherein the method further comprises:
proliferating the at least one immortalized monoclonal AMP cell line in serum-free medium, wherein the serum-free medium does not comprise EGF.

17. The method of claim 13 or 14, wherein immortalizing the isolated non-immortalized AMP cell comprises transfecting the non-immortalized AMP cell with HPV-16 E6/E7 and hTERT.

18. The method of claim 17, wherein the HPV-16 E6/E7 and hTERT are transfected via a PiggyBac transposon stable construct.

19. The method of claim 17, wherein the transfecting occurs via liposomes prepared by combining a pair of site-specific recombination sequences with a cationic lipid.

20. The method of claim 19, wherein the transfecting occurs via a liposome transfection method.

21. The method of claim 17, wherein the transfecting occurs via microspheres.

22. The method of claim 21, wherein the microspheres are prepared by combining a pair of site-specific recombination sequences with a 25% polymer solution (1:2 v/v) volumetric ratio, and incubating at 30 minutes at 70° C. followed by cooling to 23° C.

23. The method of claim 22, wherein the 25% polymer solution comprises 12.5% PVP (40,000 Daltons) and 12.5% PEG in 1M sodium acetate at pH 5.5.

24. An immortalized amnion-derived multipotent progenitor (AMP) cell line selected from clones of single cells expressing PB-E6-E7-PB-TERT.
